SEQUENCE LISTING (16 sequences)

SEQ ID NO: 1 - RNA, 21 nt, Artificial Sequence
  Description of Artificial Sequence Small inhibitory RNA (siRNA) with CD4 sense sequence.
gaucaagagacuccucaguga

SEQ ID NO: 2 - DNA, 21 bp, Artificial Sequence
  Description of Combined DNA/RNA MoleculeCD4 Antisense.
acugaggagucucuugauctg

SEQ ID NO: 3 - RNA, 21 nt, Artificial Sequence
  Description of Artificial SequenceSmall inhibitory RNA (siRNA) with p24 sense sequence.
gauuguacugagagacaggcu

SEQ ID NO: 4 - RNA, 21 nt, Artificial Sequence
  Description of Artificial SequenceSmall inhibitory RNA (siRNA) with p24 antisense sequence.
ccugucucucaguacaaucuu

SEQ ID NO: 5 - RNA, 21 nt, Artificial Sequence
  Description of Artificial SequenceSmall inhibitory RNA (siRNA) with GFP sense sequence.
ggcuacguccaggagcgcacc

SEQ ID NO: 6 - RNA, 21 nt, Artificial Sequence
  Description of Artificial SequenceSmall inhibitory RNA (siRNA) with GFP antisense sequence.
ugcgcuccuggacguagccuu

SEQ ID NO: 7 - RNA, 21 nt, Artificial Sequence
  Description of Artificial SequenceSmall inhibitory RNA (siRNA) with HPRT sense sequence.
gugucauuagugaaacuggaa

SEQ ID NO: 8 - RNA, 21 nt, Artificial Sequence
  Description of Artificial SequenceSmall inhibitory RNA (siRNA) with HPRT antisense sequence.
ccaguuucacuaaugacacaa

SEQ ID NO: 9 - DNA, 20 bp, Artificial Sequence
  Description of Artificial SequenceForward PCR primer for amplifying CD4.
tgaagtggaggaccagaagg

SEQ ID NO: 10 - DNA, 21 bp, Artificial Sequence
  Description of Artificial Sequence Reverse PCR primer for amplifying CD4.
cttgcccatctggaggcttag

SEQ ID NO: 11 - DNA, 28 bp, Artificial Sequence
  Description of Artificial SequenceForward PCT primer for amplifying p24.
ccaggggcaaatggtacatcaggccata

SEQ ID NO: 12 - DNA, 27 bp, Artificial Sequence
  Description of Artificial SequenceReverse PCT primer for amplifying p24.
cctcctgtgaagcttgctcggctctta

SEQ ID NO: 13 - DNA, 30 bp, Artificial Sequence
  Description of Artificial SequenceForward PCR primer for amplifying nef.
atgggtggcaagtggtcaaaaagtagtgtg

SEQ ID NO: 14 - DNA, 30 bp, Artificial Sequence
  Description of Artificial SequenceReverse PCR primer for amplifying nef.
gtggctaagatctacagctgccttgtaagt

SEQ ID NO: 15 - DNA, 12 bp, Artificial Sequence
  Description of Artificial Sequence There is no genetic source.  Sequences were invented for purposes of illustrating the concept of sequence complexity.
gggcccaaattt

SEQ ID NO: 16 - DNA, 12 bp, Artificial Sequence
  Description of Artificial Sequence There is no genetic source.  Sequences were invented for purposes of illustrating the concept of sequence complexity.
gtcactgctaga

(19) United States
(12) Patent Application Publication
(10) Pub. No.: US 2004/0248296 A1
(43) Pub. Date: Dec. 9, 2004

(54) HIV therapeutic

(76) Inventors: Paul J. Beresford, Aurora, IL (US); Judy Lieberman, Brookline, MA (US); Michael F. Murray, Sherborn, MA (US); Carl D. Novina, Somerville, MA (US); Phillip A. Sharp, Newton, MA (US)

Correspondence Address: Choate, Hall & Stewart, Exchange Place, 53 State Street, Boston, MA, 02109, US

(21) Appl. No.: 10/393,411

(22) Filed: Mar. 20, 2003

Related U.S. Application Data
(60) Provisional application No. 60/365,925, filed on Mar. 20, 2002.
(60) Provisional application No. 60/396,041, filed on Jul. 15, 2002.

(51) Int. Cl. A61K 48/00; C12N 5/00

(52) U.S. Cl. 435/375; 514/44

(57) ABSTRACT

The present invention provides siRNA methods and compositions for inhibiting HIV infection and/or replication, as well as systems for identifying effective siRNAs for inhibiting HIV and systems for studying HIV infective mechanisms. The invention also provides methods and compositions for inhibiting infection, pathogenicity and/or replication of an infectious agent; for example, by using siRNAs to inhibit host cell gene expression.

### CROSS-REFERENCE TO RELATED APPLICATION

This application claims priority to U.S. Provisional Patent Application 60/365,925, filed Mar. 20, 2002, and U.S. Provisional Patent Application 60/396,041, filed Jul. 15, 2002. The contents of each of these applications is incorporated herein by reference.

### GOVERNMENT SUPPORT

The United States Government has provided grant support utilized in the development of the present invention. In particular, National Cancer Institute contract number P01-CA42063, National Institutes of Health contract numbers R37-GM34277, R01-A132486, and R21-A145306 have supported development of this invention. The United States Government may have certain rights in the invention.

### BACKGROUND OF THE INVENTION

The AIDS epidemic is arguably the most devastating medical crisis humankind has confronted. Some 40 million people are infected with HIV worldwide, and new infections are occurring at the rate of 5 million per year (UNAIDS Update on the Worldwide AIDS Epidemic, December 2001). The impact of the epidemic extends far beyond the medical costs and personal losses suffered by the direct victims, as the social fabric of many countries is being strained by increased costs associated with insurance, benefits, absenteeism, illness, and training, by the sacrifices made by family members and friends struggling to care for sick loved ones, and by the loss of trained and experienced men and women who would otherwise contribute to a functional political and economic structure.

Immense amounts of time, effort, and money have been invested in pursuit of effective treatments, whether prophylactic or therapeutic, for HIV infection. In the United States, the Food and Drug Administration has approved three different classes of compounds for use in HIV therapy: nucleoside analogs, non-nucleoside reverse transcriptase inhibitors, and protease inhibitors. For many patients, combinations of these compounds have proved remarkably effective at reducing viral load, in some cases for long periods of time. Unfortunately, the therapeutic regimens are often very complex, requiring precisely orchestrated administration of multiple pills throughout the day and not tolerating even minor variation in administration. Moreover, unfortunately, many patients respond poorly to treatment even when they follow their prescribed therapeutic regimen precisely. There remains a need for the development of alternative therapies for the treatment and prevention of HIV infection and AIDS. In addition, there remains a need for the development of improved and/or alternative therapies for the treatment and prevention of a variety of other infectious diseases, e.g., diseases caused by bacteria, protozoa, fungi, and/or viruses.

### SUMMARY OF THE INVENTION

The present invention provides a novel therapeutic for the treatment of HIV. In particular, the invention provides compositions containing short interfering RNA (siRNA) targeted to one or more viral or host genes involved in viral infection and/or replication. In certain embodiments of the invention the siRNA comprises two RNA strands having a region of complementarity approximately 19 nucleotides in length and optionally further comprises one or two single-stranded overhangs or loops. In certain embodiments of the invention the siRNA comprises a single RNA strand having a region of self-complementarity. The single RNA strand may form a hairpin structure with a stem and loop and, optionally, one or more unpaired portions at the 5′ and/or 3′ portion of the RNA.

The present invention further provides methods of treating HIV infection by administering inventive siRNA-containing compositions to an infected cell or organism within an appropriate time window prior to, during, or after infection. The siRNAs may be chemically synthesized, produced using in vitro transcription, etc.

The invention provides additional methods of treating or preventing HIV infection employing gene therapy. According to certain of these methods cells (either infected or noninfected) are engineered or manipulated to synthesize inventive siRNAs. According to certain embodiments of the invention the cells are engineered to contain a construct or vector that directs synthesis of one or more siRNAs within the cell. The cells may be engineered in vitro or while present within the subject to be treated.

The present invention also provides a system for identifying siRNA compositions that are useful for the inhibition of HIV replication and/or infection.

The present invention further provides a system for the analysis and characterization of the mechanism of HIV replication and/or infection, as well as relevant viral and host components involved in the replication/infection cycle.

The invention further provides siRNA compositions targeted to host cell transcripts or agent-specific transcripts involved in infectivity, pathogenicity, or replication of various infectious agents other than HIV and also methods of treating or preventing infection by such infectious agents by administering the compositions.

This application refers to various patents, journal articles, and other publications, all of which are incorporated herein by reference.

### BRIEF DESCRIPTION OF THE DRAWINGS

FIG. 1 presents a schematic of the HIV virion and its replication cycle.

FIG. 2 shows the genome structure of HIV (FIG. 2A) and the transcripts generated from the HIV genome (FIG. 2B).

FIG. 3 shows the structure of siRNAs observed in the Drosophila system.

FIG. 4 presents a schematic representation of the steps involved in RNA interference in Drosophila.

FIG. 5 shows a variety of exemplary siRNA structures useful in accordance with the present invention.

FIG. 6 presents a representation of an alternative inhibitory pathway, in which the DICER enzyme cleaves a substrate having a base mismatch in the stem to generate an inhibitory product that binds to the 3′ UTR of a target transcript and inhibits translation.

FIG. 7 presents one example of a construct that may be used to direct transcription of both strands of an inventive siRNA.

FIG. 8 depicts one example of a construct that may be used to direct transcript of a single-stranded siRNA according to the present invention.

FIG. 9 shows the results of experiments indicating that CD4-siRNA inhibits HIV entry and infection in Magi-CCR5 cells. Panel A shows flow cytometric analysis of CD4 expression (CD4-PE) 60 hours after Magi-CCR5 cells were either mock transfected or transfected with CD4-siRNA, antisense strand of CD4-siRNA only (CD4-asRNA) or HPRT-siRNA (control siRNA). Cell numbers in each panel represent the percent of gated CD4 positive cells. Panel B shows a Northern blot for CD4 expression in control (CD4-negative) HeLa cells (lane 1), mock (lane 2), CD4-siRNA (lane 3, CD4-asRNA (lane 4) and control siRNA (lane 5) transfected cells. β-actin expression was used as a loading control. Panel C shows β-gal expression in CD4-siRNA (lane 1), CD4-asRNA (lane 2) and control siRNA (lane 3) transfected cells, 2 days after infection with HIV-1 NL43 (left) or BAL (right). A reduction in the number of β-gal positive cells in CD4-siRNA transfected cells compared with control siRNA transfected cells indicates decreased transactivation of endogenous LTR-β-gal expression by HIV-1 Tat. Error bars are the average of 2 experiments. Panel D shows a photomicrograph of β-gal stained Magi-CCR5 cells either uninfected or infected with HIV-1 NL43 after mock, CD4-siRNA, CD4-asRNA, or control siRNA transfection. Syncytia formation and LTR activation are reduced in the CD4-siRNA transfected cells compared to controls. Panel E presents levels of viral p24 antigen of cell free HIV production from the samples described in C as measured by ELISA 2 days after transfected Magi-CCR5 cells were infected with HIV-1 strains NL43 (left) or BAL (right). Error bars are the average of 2 experiments. Panel F shows alternate washes of the Northern blot shown in Panel B. The upper portion of the panel shows a lower stringency wash used for quantification of transcription after gene silencing. The middle panel is a higher stringency wash of the same blot used to demonstrate that the smudge near the CD4 silenced lane was non-specific.

FIG. 10 presents results of experiments demonstrating that p24-siRNA inhibits viral replication in HeLa-CD4 cells. Panel A shows flow cytometric analysis of p24-siRNA-directed inhibition of viral gene expression (p24RD1) in uninfected, control and mock-, p24-siRNA-, p24-siRNA-antisense strand- and GFP-siRNA (control siRNA) transfected HeLa-CD4 cells 2 d after infection with HIV_{IIIB}, demonstrating specificity of the inhibitory effect. Panel B shows a Northern blot for p24 expression in uninfected (lane 1), mock (lane 2), p24-siRNA (lane 3), p24-siRNA-antisense strand (lane 4), and control siRNA (lane 5) transfected cells. β-actin expression was used as a loading control. Panel C shows flow cytometric analysis of p24 expression (p24RD1) in uninfected control and mock, p24-siRNA and GFP-siRNA (control siRNA) transfected HeLA-CD4 cells 5 days post infection with HIV_{IIIB}. Cell numbers in each panel represent the percent of gated p24 cells. Panel B gives levels of viral p24 antigen measured by ELISA in uninfected control (lane 1) and mock (lane 2), p24-siRNA (lane 3) and control siRNA (lane 4) transfected cells infected with HIV_{IIIB} and demonstrates that reduction of cell free virus production only in p24-siRNA transfected HeLa-CD4 cells. Error bars represent the average of three experiments. Panel C is a Northern blot for p24, Nef and β-actin expression in stably infected control (lane 1), uninfected (lane 2), mock (lane 3), p24-siRNA (lane 4), and control siRNA (lane 5) transfected cells. Compared to mock or control siRNA transfected cells, p24-siRNA transfected cells showed decreased expression of the full length, 9.2 Kb HIV transcripts and/or genomic RNA as well as the 4.3 and 2.0 Kb Nef-containing transcripts. β-actin expression was used as a loading control.

FIG. 11 demonstrates siRNA-directed knockdown of viral gene expression in HeLa-CD4 cells within established HIV infection. Four days after infection with HIV_{IIIB}, HeLa-CD4 cells were either mock transfected or transfected with p24-siRNA or GFP-siRNA (control siRNA) and analyzed 2 days later for p24 expression (p24-RD1) by flow cytometry. The overlay histogram depicts the uninfected control shown in panel 1. Cell numbers in each panel depicts mean fluorescent intensity of the cells expressing p24.

FIG. 12 presents results of experiments analyzing the time course of silencing HIV gene expression and inhibition of viral replication in H9 T cells. Panel A shows flow cytometry of p24 (p24-RD1) and GFP expression in mock, GFP-siRNA, or CD19-siRNA (control siRNA) transfected H9 cells infected 24 hours later with HIV containing GFP inserted into the Nef region and analyzed 2, 5, and 9 days after transfection. Cell numbers in each panel represent the percent of cells positive for both p24 and GFP expression. Panel B shows viral p24 ELISA titers of mock (lane 1), GFP-siRNA (lane 2), or control siRNA (lane 3) at 2, 5, and 9 days after infection.

FIG. 13 shows a model for pathways of RNA interference for inhibition of productive HIV infection. siRNA directed to the viral receptor inhibits virus entry into target cells (Step 1). Silencing of pre-integrated HIV may occur by p24-siRNA targeting the RISC complex directly to the HIV genome to prevent integration (Step 2). In addition, HIV progeny virus production may be inhibited by silencing full length HIV gene expression (mRNA or genomic RNA) expressed from the integrated provirus (Step 3).

FIG. 14 presents results of an experiment demonstrating siRNA-directed silencing of viral gene expression after HIV integration. ACH2 cells were mock-transfected and left uninduced or mock-transfected or transfected with p24-siRNA or with GFP-siRNA (control siRNA) and induced with PMA. The samples were analyzed 2 days after induction for p24 expression (p24-RD1) by flow cytometry. Numbers in each panel represent percent of cells expressing p24. Note the different scale for p24-siRNA transfected cells.

FIG. 15 presents results from an experiment demonstrating siRNA-directed silencing of viral gene expression in primary T cells. CD4⁺cells activated with PHA for 4 days were mock, p24-siRNA, or GFP-siRNA (control siRNA) transfected. Twenty four hours later, the CD4⁺blasts were infected with HIV_{IIIB}. Cells were analyzed 2 days later for p24 expression (p24-RD1) by flow cytometry. Cell numbers in each panel represent the percent of cells positive for p24 expression.

### DEFINITIONS

The term hybridize, as used herein, refers to the interaction between two complementary nucleic acid sequences. The phrase hybridizes under high stringency conditions describes an interaction that is sufficiently stable that it is maintained under art-recognized high stringency conditions. Guidance for performing hybridization reactions can be found, for example, in Current Protocols in Molecular Biology, John Wiley & Sons, N.Y., 6.3.1-6.3.6, 1989, and more recent updated editions, all of which are incorporated by reference. See also Sambrook, Russell, and Sambrook, Molecular Cloning: A Laboratory Manual, 3^{rd} ed., Cold Spring Harbor Laboratory Press, Cold Spring Harbor, 2001. Aqueous and nonaqueous methods are described in that reference and either can be used. Typically, for nucleic acid sequences over approximately 50-100 nucleotides in length, various levels of stringency are defined, such as low stringency (e.g., 6× sodium chloride/sodium citrate (SSC) at about 45° C., followed by two washes in 0.2×SSC, 0.1% SDS at least at 50° C. (the temperature of the washes can be increased to 55° C. for medium-low stringency conditions)); 2) medium stringency hybridization conditions utilize 6×SSC at about 45° C., followed by one or more washes in 0.2×SSC, 0.1% SDS at 60° C.; 3) high stringency hybridization conditions utilize 6×SSC at about 45° C., followed by one or more washes in 0.2×SSC, 0.1% SDS at 65° C.; and 4) very high stringency hybridization conditions are 0.5M sodium phosphate, 0.1% SDS at 65° C., followed by one or more washes at 0.2×SSC, 1% SDS at 65° C.) Hybridization under high stringency conditions only occurs between sequences with a very high degree of complementarity. One of ordinary skill in the art will recognize that the parameters for different degrees of stringency will generally differ based various factors such as the length of the hybridizing sequences, whether they contain RNA or DNA, etc. For example, appropriate temperatures for high, medium, or low stringency hybridization will generally be lower for shorter sequences such as oligonucleotides than for longer sequences.

The term human immunodeficiency virus (HIV), is used here to refer to any strain of HIV-1 or HIV-2 that is capable of causing disease in a human subject, or that is an interesting candidate for experimental analysis. Furthermore, as will be clear from context, the term HIV is often used to refer to a virus (e.g., FIV, SIV) that is highly related to HIV but infects a different host. A huge number of HIV and SIV isolates have been partially or completely sequenced; Appendix A presents merely a representative list of HIV and SIV clones whose complete sequence has been deposited in a public database (Genbank; search was done on Mar. 20, 2002). Sequences of HIV genes are therefore readily available to, or determinable by, those of ordinary skill in the art.

Isolated, as used herein, means 1) separated from at least some of the components with which it is usually associated in nature; and/or 2) not occurring in nature.

Purified, as used herein, means separated from many other compounds or entities. A compound or entity may be partially purified, substantially purified, or pure, where it is pure when it is removed from substantially all other compounds or entities, i.e., is preferably at least about 90%, more preferably at least about 91%, 92%, 93%, 94%, 95%, 96%, 97%, 98%, 99%, or greater than 99% pure.

The term regulatory sequence or regulatory element is used herein to describe a region of nucleic acid sequence that directs, enhances, or inhibits the expression (particularly transcription, but in some cases other events such as splicing or other processing) of sequence(s) with which it is operatively linked. The term includes promoters, enhancers and other transcriptional control elements. In some embodiments of the invention, regulatory sequences may direct constitutive expression of a nucleotide sequence; in other embodiments, regulatory sequences may direct tissue-specific and/or inducible expression. For instance, non-limiting examples of tissue-specific promoters appropriate for use in mammalian cells include lymphoid-specific promoters (see, for example, Calame et al., Adv. Immunol. 43:235, 1988) such as promoters of T cell receptors (see, e.g., Winoto et al., EMBO J 8:729, 1989) and immunoglobulins (see, for example, Banerji et al., Cell 33:729, 1983; Queen et al., Cell 33:741, 1983), and neuron-specific promoters (e.g., the neurofilament promoter; Byrne et al., Proc. Natl. Acad. Sci. USA 86:5473, 1989). Developmentally-regulated promoters are also encompassed, including, for example, the murine hox promoters (Kessel et al., Science 249:374, 1990) and the α-fetoprotein promoter (Campes et al., Genes Dev. 3:537, 1989). In some embodiments of the invention regulatory sequences may direct expression of a nucleotide sequence only in cells that have been infected with an infectious agent. For example, the regulatory sequence may comprise a promoter and/or enhancer such as a virus-specific promoter or enhancer that is recognized by a viral protein, e.g., a viral polymerase, transcription factor, etc.

A short, interfering RNA (siRNA) comprises an RNA duplex that is approximately 19 basepairs long and optionally further comprises one or two single-stranded overhangs or loops. An inventive siRNA may comprise two RNA strands hybridized together, or may alternatively comprise a single RNA strand that includes a self-hybridizing portion. When siRNAs utilized in accordance with the present invention include one or more free strand ends, it is generally preferred that free 5′ ends have phosphate groups, and free 3′ ends have hydroxyl groups. Inventive siRNAs include a portion that hybridizes under stringent conditions with a target transcript. In certain preferred embodiments of the invention, one strand of the siRNA (or, the self-hybridizing portion of the siRNA) is precisely complementary with a region of the target transcript, meaning that the siRNA hybridizes to the target transcript without a single mismatch. In most embodiments of the invention in which perfect complementarity is not achieved, it is generally preferred that any mismatches be located at or near the siRNA termini.

The term subject, as used herein, refers to an individual susceptible to infection with an infectious agent, e.g., an individual susceptible to infection with an immunodeficiency virus such as HIV. Preferred subjects are mammals, particularly domesticated mammals (e.g., dogs, cats, etc.), primates, or humans.

An siRNA is considered to be targeted for the purposes described herein if 1) the stability of the target gene transcript is reduced in the presence of the siRNA as compared with its absence; and/or 2) the siRNA shows at least about 90%, more preferably at least about 91%, 92%, 93%, 94%, 95%, 96%, 97%, 98%, 99%, or 100% precise sequence complementarity with the target transcript for a stretch of at least about 17, more preferably at least about 18 or 19 to about 21-23 nucleotides; and/or 3) the siRNA hybridizes to the target transcript under stringent conditions.

The term vector is used herein to refer to a nucleic acid molecule capable of mediating entry of, e.g., transferring, transporting, etc., another nucleic acid molecule into a cell. The transferred nucleic acid is generally linked to, e.g., inserted into, the vector nucleic acid molecule. A vector may include sequences that direct autonomous replication, or may include sequences sufficient to allow integration into host cell DNA. Useful vectors include, for example, plasmids, cosmids, and viral vectors. Viral vectors include, e.g., replication defective retroviruses, adenoviruses, adeno-associated viruses, and lentiviruses. As will be evident to one of ordinary skill in the art, viral vectors may include various viral components in addition to nucleic acid(s) that mediate entry of the transferred nucleic acid. The present invention provides vectors from which siRNAs may be expressed in relevant expression systems, e.g., cells. Preferably, such expression vectors include one or more regulatory sequences operatively linked to the nucleic acid sequence(s) to be expressed.

### DETAILED DESCRIPTION OF CERTAIN PREFERRED EMBODIMENTS OF THE INVENTION

siRNA Compositions

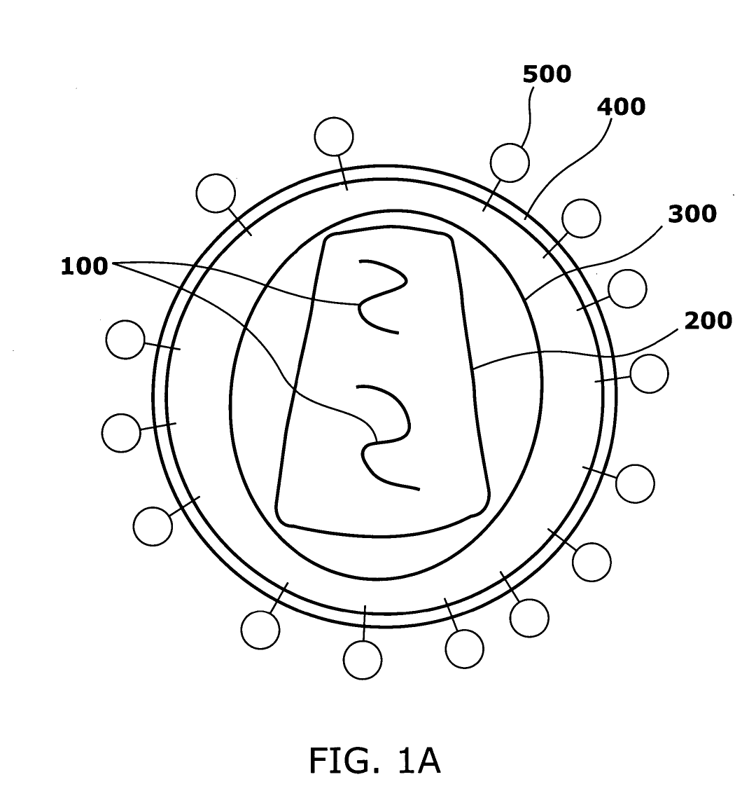

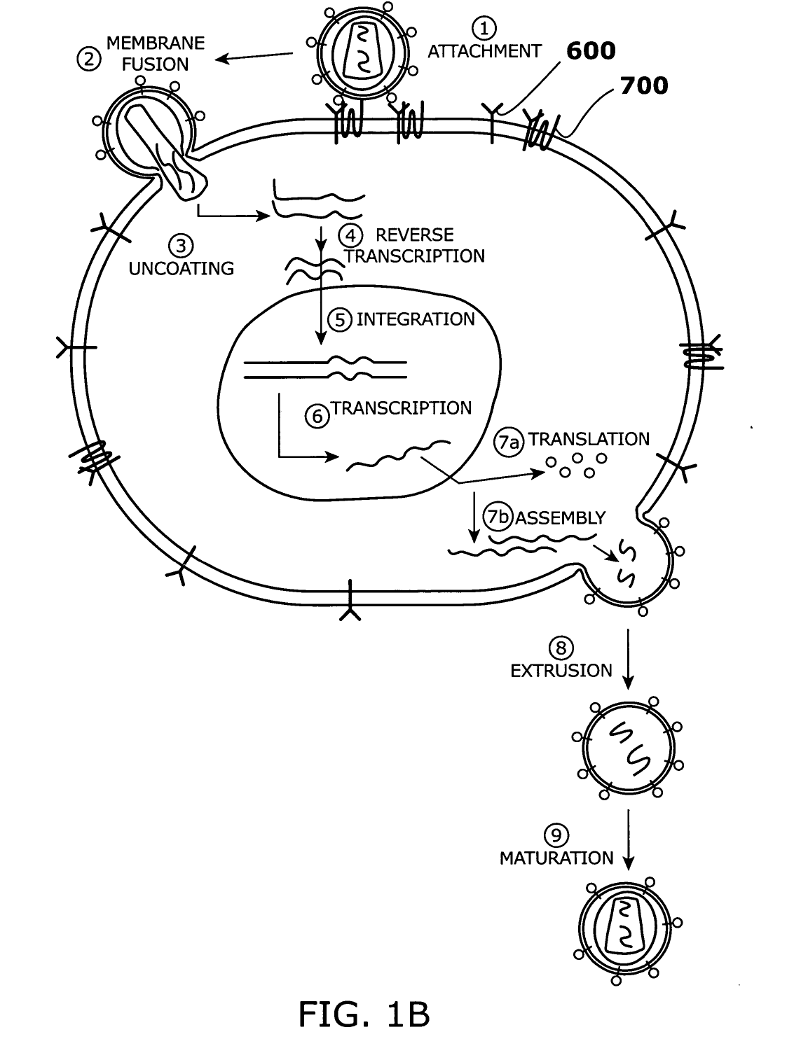

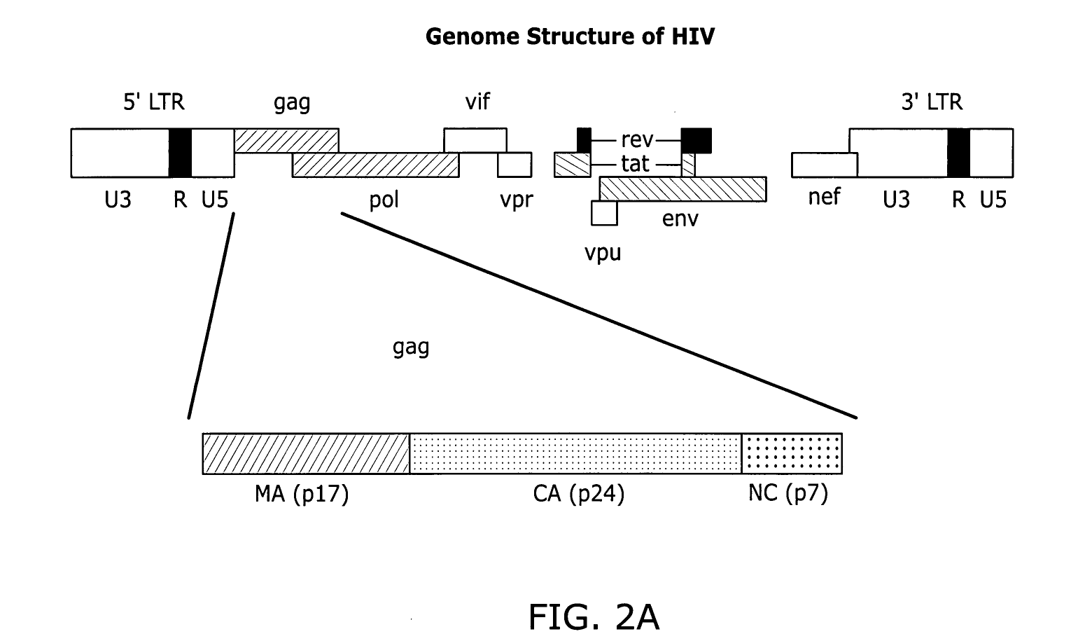

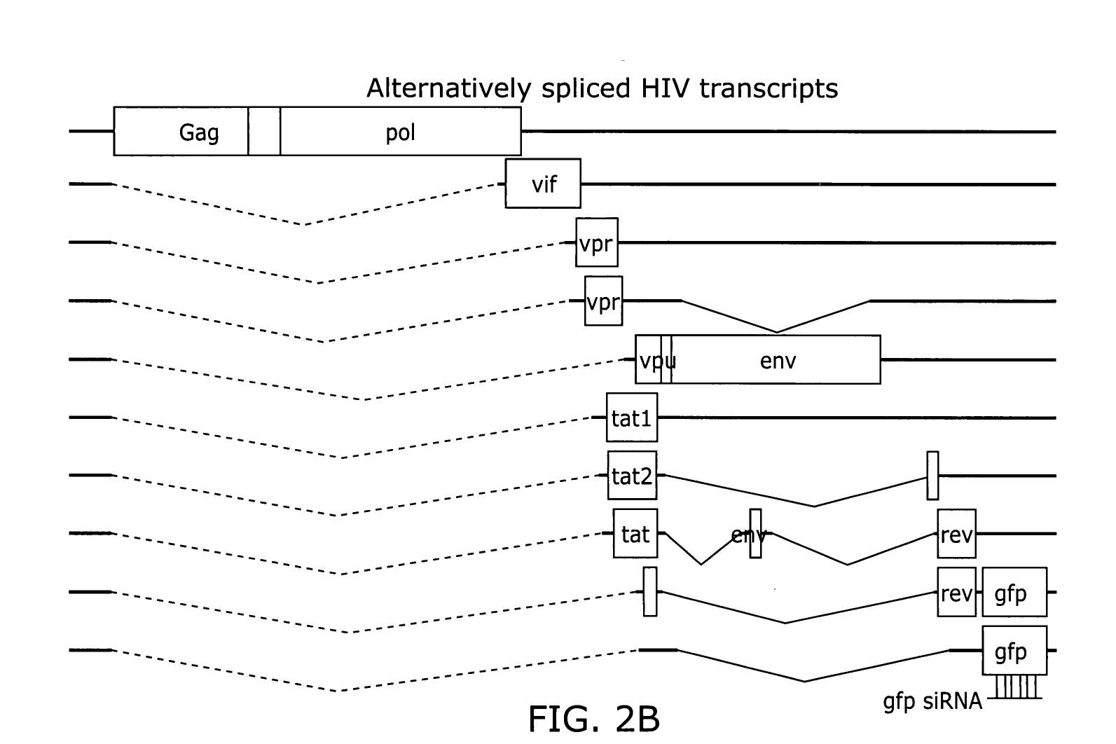

As indicated above, the present invention provides compositions containing siRNA(s) targeted to one or more viral or host gene(s) involved in HIV infection and/or replication. The HIV infection/replication cycle is depicted schematically in FIG. 1. As shown in FIG. 1A, the HIV virion comprises two copies of the HIV genome 100 packaged inside a p24 protein capsid 200 which is encased by a p17 protein matrix 300 that in turn is surrounded by a lipid bilayer 400 from which the extracellular domain 500 of the envelope glycoprotein gp120 protrudes. The infective cycle (FIG. 1B) begins when the HIV virion attaches to the surface of a susceptible cell through interaction of gp120 with the cell surface receptor CD4 600 and a co-receptor 700, resulting in membrane fusion. As the virus fuses with the cell, the viral core is injected into the cytoplasm, where the matrix and capsid become dismantled so that the viral genome (FIG. 2) is released into the cytoplasm. A viral reverse transcriptase then copies the RNA genome into DNA, and this DNA moves into the nucleus, assisted by the viral vpr and MA proteins, where its integration into host cell DNA is catalyzed by the integrase enzyme.

Once integrated into a host genome, viral DNA can remain dormant for very long periods of time, possibly even for years. When activated, the viral DNA is transcribed by host cell RNA polymerase, so that a 9 Kb genomic transcript is generated. This 9 Kb transcript is both a genome for a new virion and a transcript from which the viral gag (p55) and gag-pol (p160) polyproteins are synthesized. These polyproteins are later processed into the matrix (MA), capsid (CA), and nucleocapsid (NC) proteins (in the case of gag), or the matrix, capsid, proteinase (PR), reverse transcriptase (RT), and integrase (INT) proteins (in the case of gag-pol). The full-length 9 Kb viral RNA transcript also is spliced to yield various other transcripts, including 4 Kb and 2 Kb products, that act as templates for the synthesis of other viral proteins. The 4 Kb transcript is translated to produce gp160, which will be processed into the gp120 and gp41 envelope glycoproteins, and also the regulating proteins vif, vpr, and vpu; the 2 Kb transcript is translated to produce tat, rev, and nef (for a discussion of various transcripts present at different times during the HIV life cycle, see, for example, Kim et al., J. Virol. 63:3708, 1989, incorporated herein by reference).

Newly made gag and gag-pol polyproteins associate with one another, with complete viral genomes, and with gp41 in the cell membrane so that a new viral particle begins to assemble at the membrane. As assembly continues, the structure extrudes from the cell, thereby acquiring a lipid coat punctuated with envelope glycoproteins. After the immature virion is released from the cell, it matures through the action of the viral protease on the gag and gag-pol polyproteins, which releases the active structural proteins matrix and capsid, etc.

The complex interactions of host and viral proteins involved in the HIV life cycle offer a variety of targets for anti-HIV therapy with siRNA according to the present invention. For example, siRNAs that target host proteins such as the receptor or co-receptor could inhibit viral binding and cell entry. siRNAs that target other host proteins, including RNA polymerase II or the protease that cleaves gp160 into gp120 and gp41 could significantly interfere with later stages of the viral life cycle. siRNAs that target viral genes could reduce the amount of 9 Kb transcript present in cells, resulting in a reduction in the number of virions that can be assembled, as well as a reduction in the amounts of other viral transcripts and the proteins encoded by them. Of course, siRNAs that target viral genes will also specifically reduce the level of either the 4 Kb or 2 Kb transcript, or of other transcripts that include the targeted sequence.

Thus, according to the present invention, potential cellular transcripts that could be targets for siRNA therapy include, but are not limited to, transcripts for 1) the CD4 receptor; 2) any of the variety of chemokine receptors utilized by HIV strains (e.g., CXCR4, CCR5, CCR3, CCR2, CCR1, CCR4, CCR8, CCR9, CXCR2, STRL33, US28, V28, gpr1, gpr15, Apj, ChemR23, etc); 3) other cell surface molecules that may participate in viral entry (e.g., CD26, VPAC1, etc.), or proteins that produce such cell surface molecules (e.g., enzymes that synthesize heparan sulfate proteoglycans, galactoceramides, etc.); 4) cellular enzymes that participate in the viral life cycle (e.g., RNA polymerase II, N-myristoyltransferase, glycosylation enzymes, gp160-processing enzymes, ribonucleotide reductase, enzymes involved in polyamine biosynthesis, proteins involved in viral budding such as TSG101, etc.); 5) cellular transcription factors (e.g., Sp1, NFκB, etc.); 6) cytokines and second messengers (e.g., TNFα, IL-1α, IL-6, phospholipase C, protein kinase C, proteins involved in regulating intracellular calcium); 7) cellular accessory molecules (e.g., cyclophilins, MAP-kinase, ERK-kinase, etc.). As is evident from the foregoing description, appropriate targets include any host cell RNA or protein involved in any stage or aspect of the viral life cycle, e.g., RNAs or proteins involved in viral fusion, entry, reverse transcription, integration, transcription, replication, assembly, budding, infectivity, virulence, and/or pathogenicity.

Potential viral transcripts that could serve as a target for siRNA therapy according to the present invention include, for example, 1) the HIV genome (including the viral LTR); 2) transcripts for any viral proteins including capsid (CA, p24), matrix (MA, p17), the RNA binding proteins p9 and p7, the other gag proteins p6, p2, and p1, polymerase (p61, p55), reverse transcriptase, RNase H, protease (p10), integrase (p32), envelope (p160, p120, and/or p41), tat, rev, nef, vif, vpr, vpu, and/or vpx. See Greene, W. and Peterlin, M., Nature Medicine, 8(7), pp. 673-680, 2002, and references therein for additional discussion of host and viral genes and their roles in the viral life cycle.

Particularly preferred targets for inventive siRNA therapy are transcripts that are not required for essential activities of cells. For instance, RNA polymerase II is essential to host cell viability and therefore is not an ideal target for inventive siRNA therapy. By contrast, the CD4 receptor, the co-receptors, and any or all viral proteins are not generally considered to be essential for cell viability. That notwithstanding, the CD4 receptor is involved in a variety of important cellular functions. Some co-receptors may also be important, or even essential, in particular cell types and/or at during particular stages of development. For example, the CXCR4 receptor is apparently required for proper vascularization and may be essential at early stages of development, as studies in transgenic mice show that disruption of CXCR4 results in embryonic lethality (Tachibana et al., Nature 393:591, 1998). Nevertheless, such molecules may be preferred targets for siRNA therapy since their important or essential role may be limited to early developmental stages, and their activity may be dispensable in developed or adult organisms. In general, viral transcripts and also host cell transcripts that encode molecules whose activity is not important or essential in the cell and/or organism to which siRNA is delivered, are particularly preferred targets for siRNA therapy according to the present invention. Such host cell transcripts include the CCR5 co-receptor transcript.

Whatever gene target is selected, the design of siRNAs for use in accordance with the present invention will preferably follow certain simple guidelines. In general, it will be desirable to target sequences that are specific to the virus (as compared with the host), and that, preferably, are important or essential for viral function. Although the HIV virus is characterized by a high mutation rate and is capable of tolerating mutations, those of ordinary skill in the art will appreciate that certain regions and/or sequences tend to be conserved; such sequences may be particularly effective targets. Those of ordinary skill in the art can readily identify such conserved regions through review of the literature and/or comparisons of HIV gene sequences, a large number of which are publicly available (see, for example, Exhibit A). Also, in many cases, the agent that is delivered to a cell according to the present invention may undergo one or more processing steps before becoming an active suppressing agent (see below for further discussion); in such cases, those of ordinary skill in the art will appreciate that the relevant agent will preferably be designed to include sequences that may be necessary for its processing. In general we have found that a significant portion (generally greater than about half) of the sequences we select using these design parameters prove to be efficient suppressing sequences when included in an siRNA and tested as described herein.

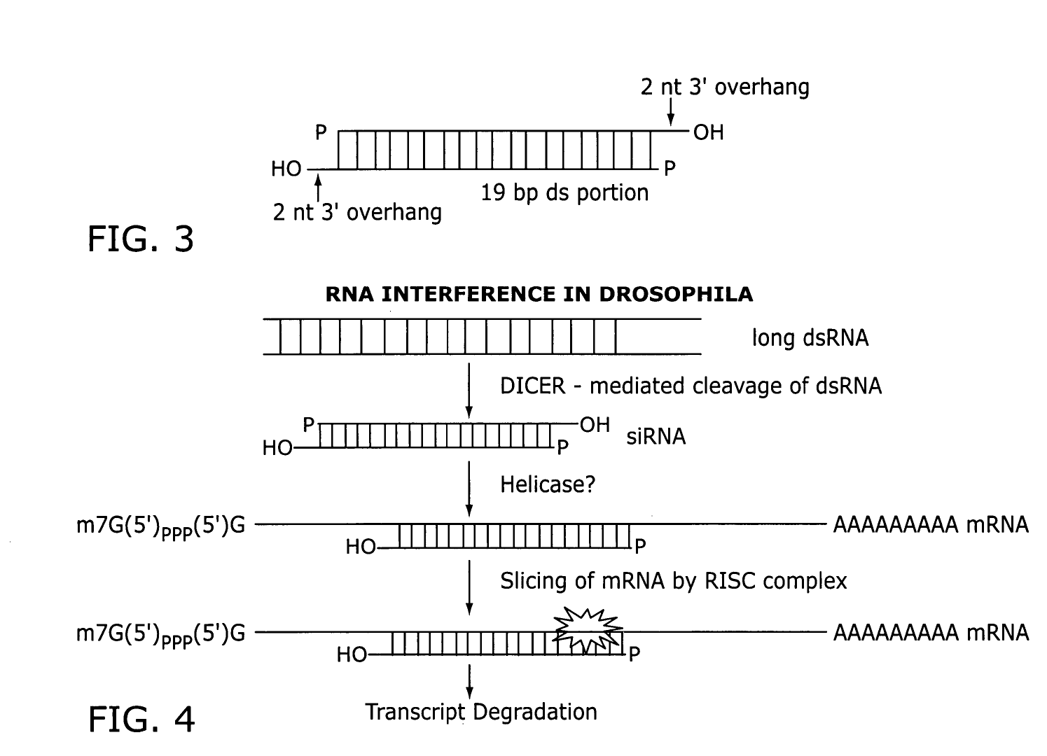

For instance, small inhibitory RNAs were first discovered in studies of the phenomenon of RNA interference (RNAi) in Drosophila, as described in WO 01/75164. In particular, it was found that, in Drosophila, long double-stranded RNAs are processed by an RNase III-like enzyme called DICER (Bernstein et al., Nature 409:363, 2001) into smaller dsRNAs comprised of two 21 nt strands, each of which has a 5′ phosphate group and a 3′ hydroxyl, and includes a 19 nt region precisely complementary with the other strand, so that there is a 19 nt duplex region flanked by 2 nt-3′ overhangs (see FIG. 3). These small dsRNAs (siRNAs) act to silence expression of any gene that includes a region complementary to one of the dsRNA strands, presumably because a helicase activity unwinds the 19 bp duplex in the siRNA, allowing an alternative duplex to form between one strand of the siRNA and the target transcript. This new duplex then guides an endonuclease complex, RISC, to the target RNA, which it cleaves (“slices”) at a single location, producing unprotected RNA ends that are promptly degraded by cellular machinery (see FIG. 4).

Homologs of the DICER enzyme have now been found in diverse species ranging from E. coli to humans (Sharp, Genes Dev. 15;485, 2001; Zamore, Nat. Struct. Biol. 8:746, 2001), raising the possibility that an RNAi-like mechanism might be able to silence gene expression in a variety of different cell types including mammalian, or even human, cells. Unfortunately, however, long dsRNAs (e.g., dsRNAs having a double-stranded region longer than about 30 nucleotides) are known to activate the interferon response in mammalian cells. Thus, rather than achieving the specific gene silencing observed with the Drosophila RNAi mechanism depicted in FIG. 4, introduction of long dsRNAs into mammalian cells would lead to interferon-mediated non-specific suppression of translation, potentially resulting in cell death. Long dsRNAs are therefore not thought to be useful for inhibiting expression of particular genes in mammalian cells.

On the other hand, we have found that siRNAs, when introduced into mammalian cells, can effectively reduce the expression of host genes and/or viral genes. In particular, we show that an siRNA targeted to human CD4 reduces the amount of CD4 mRNA and protein produced in human cells (Example 1). We also show that an siRNA targeted to the HIV p24 gene reduces the levels of p24 protein, and also reduces the levels of a variety of viral transcripts (Example 3). Moreover, we have found that these siRNAs are also capable of suppressing HIV entry, infection, and/or replication (Examples 1-4). These effects have been demonstrated in cell lines, including cell lines that are latently infected with HIV, and also in primary cells. Thus, the present invention demonstrates that treatment with siRNA is an effective strategy for inhibiting HIV infection and/or replication.

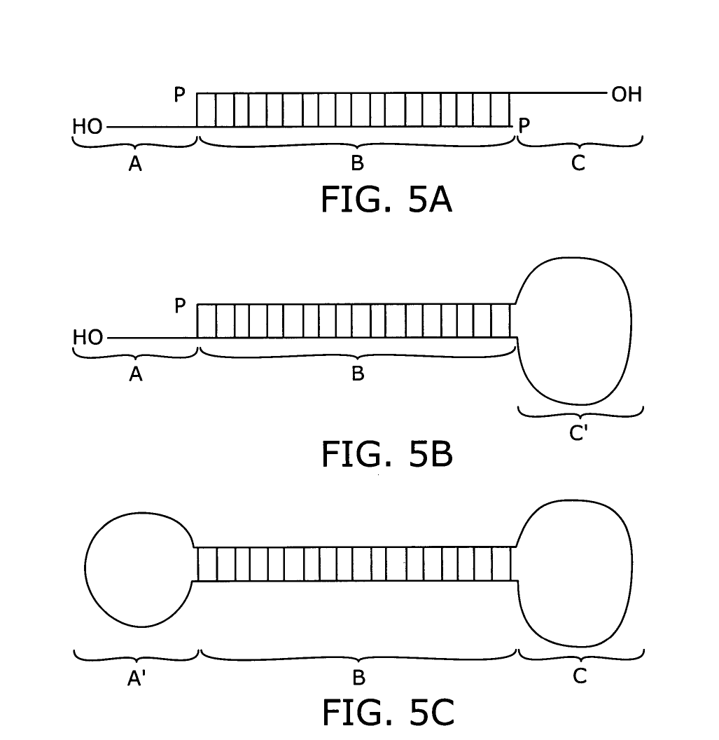

Preferred siRNAs for use in accordance with the present invention include a base-paired region approximately 19 nt long, and may optionally have free or looped ends. For example, FIG. 5 presents various structures that could be utilized as siRNAs according to the present invention. FIG. 5A shows the structure found to be active in the Drosophila system described above, and may represent the species that is active in mammalian cells; the present invention encompasses administration of an siRNA having the structure depicted in FIG. 5A to mammalian cells in order to treat or prevent HIV infection. However, it is not required that the administered agent have this structure. For example, the administered composition may include any structure capable of being processed in vivo to the structure of FIG. 5A, so long as the administered agent does not induce other negative events such as induction of the interferon response. The invention may also comprise administration of agents that are not processed to precisely the structure depicted in FIG. 5A, so long as administration of such agents reduces host or viral transcript levels sufficiently as discussed herein. FIGS. 5B and 5C present two alternative structures for use as siRNAs in accordance with the present invention.

FIG. 5B shows an agent comprising an RNA strand containing two complementary elements that hybridize to one another to form a stem (element B), a loop (element C), and an overhang (element A). Preferably, the stem is approximately 19 bp long, the loop is about 1-20, more preferably about 4-10, and most preferably about 6-8 nt long and/or the overhang is about 1-20, and more preferably about 2-15 nt long. In certain embodiments of the invention the stem is minimally 19 nucleotides in length and may be up to approximately 29 nucleotides in length. One of ordinary skill in the art will appreciate that loops of 4 nucleotides or greater are less likely subject to steric constraints than are shorter loops and therefore may be preferred. In some embodiments, the overhang includes a 5′ phosphate and a 3′ hydroxyl. As discussed below, an agent having the structure depicted in FIG. 5B can readily be generated by in vivo or in vitro transcription; in several preferred embodiments, the transcript tail will be included in the overhang, so that often the overhang will comprise a plurality of U residues, e.g., between 1 and 5 U residues. It is noted that synthetic siRNAs that have been studied in mammalian systems often have 2 overhanging U residues.

FIG. 5C shows an agent comprising an RNA circle that includes complementary elements sufficient to form a stem approximately 19 bp long (element B). Such an agent may show improved stability as compared with various other siRNAs described herein.

It will be appreciated by those of ordinary skill in the art that agents having any of the structures depicted in FIG. 5, or any other effective structure as described herein, may be comprised entirely of natural RNA nucleotides, or may instead include one or more nucleotide analogs. A wide variety of such analogs is known in the art; the most commonly-employed in studies of therapeutic nucleic acids being the phosphorothioate (for some discussion of considerations involved when utilizing phosphorothioates, see, for example, Agarwal, Biochim. Biophys. Acta 1489:53, 1999). In particular, in certain embodiments of the invention it may be desirable to stabilize the siRNA structure, for example by including nucleotide analogs at one or more free strand ends in order to reduce digestion, e.g., by exonucleases. The inclusion of deoxynucleotides, e.g., pyrimidines such as deoxythymidines at one or more free ends may serve this purpose. Alternatively or additionally, it may be desirable to include one or more nucleotide analogs in order to increase or reduce stability of the 19 bp stem, in particular as compared with any hybrid that will be formed by interaction of one strand of the siRNA with a target transcript.

Numerous nucleotide analogs and nucleotide modifications are known in the art, and their effect on properties such as hybridization and nuclease resistance has been explored. For example, various modifications to the base, sugar and internucleoside linkage have been introduced into oligonucleotides at selected positions, and the resultant effect relative to the unmodified oligonucleotide compared. A number of modifications have been shown to alter one or more aspects of the oligonucleotide such as its ability to hybridize to a complementary nucleic acid, its stability, etc. For example, useful 2′-modifications include halo, alkoxy and allyloxy groups. U.S. Pat. Nos. 6,403,779; 6,399,754; 6,225,460; 6,127,533; 6,031,086; 6,005,087; 5,977,089, and references therein disclose a wide variety of nucleotide analogs and modifications that may be of use in the practice of the present invention. See also Crooke, S. (ed.) Antisense Drug Technology: Principles, Strategies, and Application (1^{st} ed), Marcel Dekker; ISBN: 0824705661; 1st edition (2001) and references therein. As will be appreciated by one of ordinary skill in the art, analogs and modifications may be tested using, e.g., the assays described herein or other appropriate assays, in order to select those that effectively reduce expression of host and/or viral genes.

In certain embodiments of the invention the analog or modification results in an siRNA with increased oral absorbability, increased stability in the blood stream, increased ability to cross cell membranes, etc. As will be appreciated by one of ordinary skill in the art, analogs or modifications may result in altered Tm, which may result in increased tolerance of mismatches between the siRNA sequence and the target while still resulting in effective suppression.

It will further be appreciated by those of ordinary skill in the art that effective siRNA agents for use in accordance with the present invention may comprise one or more moieties that is/are not nucleotides or nucleotide analogs.

In general, inventive siRNAs will preferably include a region (the “inhibitory region”) that is substantially complementary to that found in a portion of the target transcript, so that a precise hybrid can form in vivo between one strand of the siRNA and the target transcript. Preferably, this substantially complementary region includes most or all of the stem structure depicted in FIG. 5. In certain preferred embodiments of the invention, the relevant inhibitor region of the siRNA is perfectly complementary with the target transcript; in other embodiments, one or more non-complementary residues are located at or near the ends of the siRNA/template duplex. As will be appreciated by those of ordinary skill in the art, it is generally preferred that mismatches in the central portion of the siRNA/template duplex be avoided (see, for example, Elbashir et al., EMBO J 20:6877, 2001, incorporated herein by reference).

In preferred embodiments of the invention, the siRNA hybridizes with a target site that includes exonic sequences in the target transcript. Hybridization with intronic sequences is not excluded, but generally appears not to be preferred in mammalian cells. In certain preferred embodiments of the invention, the siRNA hybridizes exclusively with exonic sequences. In some embodiments of the invention, the siRNA hybridizes with a target site that includes only sequences within a single exon; in other embodiments the target site is created by splicing or other modification of a primary transcript. Any site that is available for hybridization with an siRNA resulting in slicing and degradation of the transcript may be utilized in accordance with the present invention. Nonetheless, those of ordinary skill in the art will appreciate that, in some instances, it may be desirable to select particular regions of target gene transcript as siRNA hybridization targets. For example, it may be desirable to avoid sections of target gene transcript that may be shared with other transcripts whose degradation is not desired.

Alternatively or additionally, it may be desirable to avoid target sites that include long strings (e.g., longer than three in a row) of a single nucleotide, which therefore might allow an siRNA to hybridize inaccurately. Similarly, it may be desirable to utilize high complexity target sites, e.g., sites that include most or all residues, preferably in a stochastic pattern, avoiding stretches in which a single residue is repeated multiple times. For example, even though the sequences GGGCCCAAATTT (SEQ ID NO:15) and GTCACTGCTAGA (SEQ ID NO:16) both contain 3 G residues, 3 C residues, 3 A residues, and 3 T residues, the second sequence exhibits greater complexity than the first since it lacks contiguous blocks of G, C, A, or T. In addition, it will often be desirable to select a target site so that the ratio of GC to AU basepairs in the siRNA/template duplex is within the range of approximately 0.75:1 to approximately 1.25:1, preferably within the range of approximately 0.9:1 to approximately 1.1:1, more preferably closer to approximately or exactly 1:1. It may further be desirable to select a target site so that individual nucleotides are represented on both strands of the siRNA/template duplex, preferably approximately equally. According to the present invention, it will often be desirable to utilize siRNAs that hybridize within the 3′ half of the target transcript, as we find that selection of a target site near the 3′ end often results in better gene silencing as compared with selection of a target site elsewhere in a transcript.

One approach to selecting appropriate target sites proceeds as follows: First, the target transcript is converted into the corresponding double-stranded DNA format. The sequence is scanned to identify stretches of 19 nucleotides in which either one or both of the two nucleotides following the 3′ terminus of the 19 nucleotide stretch on each strand is a pyrimidine. Preferably the nucleotide at the 3′ terminus of both 21 nucleotide strands is a pyrimidine. The 19 nucleotide stretch is then evaluated with respect to its nucleotide composition and complexity as outlined above. Preferred sequences do not contain stretches of 3 or more identical nucleotides (e.g., GGG, CCC, AAA, TTT) on either strand. When the sequence is displayed on paper or on a screen it may be convenient to use a device such as a piece of paper in which is cut a “window” whose size corresponds to a 19 nucleotide double-stranded region with 2 nucleotide extensions at the 3′ ends. The window allows the eye to readily focus on portions of the sequence that have the appropriate size and configuration. The above method may readily be modified to identify candidate siRNAs having a double-stranded region with a length other than 19 base pairs and/or 3′ overhangs with lengths other than 2 nucleotides. In general, coding regions and regions closer to the 3′ end of the transcript than to the 5′ end are preferred. While not wishing to be bound by any theory, the inventors suggest that the 3′ portion of target transcripts may be less likely to exhibit secondary structure that may inhibit or interfere with siRNA activity, e.g., by reducing accessibility.

One of ordinary skill in the art will appreciate that siRNAs may exhibit a range of melting temperatures (Tm) and dissociation temperatures (Td) in accordance with the foregoing principles. The Tm is defined as the temperature at which 50% of a nucleic acid and its perfect complement are in duplex in solution while the Td, defined as the temperature at a particular salt concentration, and total strand concentration at which 50% of an oligonucleotide and its perfect filter-bound complement are in duplex, relates to situations in which one molecule is immobilized on a filter. Representative examples of acceptable Tms may readily be determined using methods well known in the art, either experimentally or using appropriate empirically or theoretically derived equations, based on the siRNA sequences disclosed in the Examples herein. One common way to determine the actual Tm is to use a thermostatted cell in a UV spectrophotometer. If temperature is plotted vs. absorbance, an S-shaped curve with two plateaus will be observed. The absorbance reading halfway between the plateaus corresponds to Tm. The simplest equation for Td is the Wallace rule: Td=2(A+T)+4(G+C) Wallace, R. B.; Shaffer, J.; Murphy, R. F.; Bonner, J.; Hirose, T.; Itakura, K., Nucleic Acids Res. 6, 3543 (1979). The nature of the immobilized target strand provides a net decrease in the Tm observed relative to the value when both target and probe are free in solution. The magnitude of the decrease is approximately 7-8° C. Another useful equation for DNA which is valid for sequences longer than 50 nucleotides from pH 5 to 9 within appropriate values for concentration of monovalent cations, is: Tm=81.5+16.6 log M+41(XG+XC)−500/L−0.62F, where M is the molar concentration of monovalent cations, XG and XC are the mole fractions of G and C in the sequence, L is the length of the shortest strand in the duplex, and F is the molar concentration of formamide (Howley, P. M; Israel, M. F.; Law, M-F.; Martin, M. A., J. Biol. Chem. 254,4876). Similar equations for RNA are: Tm=79.8+18.5 log M+58.4(XG+XC)+11.8(XG+XC)2−820/L−0.35F and for DNA-RNA hybrids: Tm=79.8+18.5 log M+58.4(XG+XC)+11.8(XG+XC)2−820/L−0.50F. These equations are derived for immobilized target hybrids. Several studies have derived accurate equations for Tm using thermodynamic basis sets for nearest neighbor interactions. The equation for DNA and RNA is: Tm=(1000ΔH)/A+AS+Rln(Ct/4)−273.15+16.6 ln[Na⁺], where ΔH (Kcal/mol) is the sum of the nearest neighbor enthalpy changes for hybrids, A (eu) is a constant containing corrections for helix initiation, ΔS (eu) is the sum of the nearest neighbor entropy changes, R is the Gas Constant (1.987 cal deg⁻¹ mol⁻¹) and Ct is the total molar concentration of strands. If the strand is self complementary, Ct/4 is replaced by Ct. Values for thermodynamic parameters are available in the literature. For DNA see Breslauer, et al., Proc. Natl. Acad. Sci. USA 83, 3746-3750, 1986. For RNA:DNA duplexes see Sugimoto, N., et al, Biochemistry, 34(35): 11211-6, 1995. For RNA see Freier, S.M., et al., Proc. Natl. Acad. Sci. 83, 9373-9377, 1986. Rychlik, W., et al., Nucl. Acids Res. 18(21), 6409-6412, 1990. Various computer programs for calculating Tm are widely available. See, e.g., the Web site having URL www.basic.nwu.eduibiotools/oligocalc.html.

The accessibility of various portions of a target transcript may be assessed using RNase H protection techniques, taking advantage of the ability of RNase H to selectivly cleave the RNA portion of RNA/DNA hybrids. In one such assay, oligonucleotides having the sequence of either strand of a candidate siRNA are allowed to hybridize to target RNA transcripts. The target transcript is exposed to RNase H under conditions compatible with RNase H activity. If the oligonucleotide is able to anneal to the complementary sequence of the RNA, RNase H will cleave the RNA within the double-stranded DNA/RNA region. However, regions of the target RNA that are capable of forming secondary structures, e.g., self-complementary regions, are more likely to be resistant to RNase H digestion than regions that do not form such structures. Portions of the RNA that survive such exposure are isolated and sequenced. These portions represent sequence that may be less accessible and thus not preferred for the design of siRNAs. RNA to be tested may be chemically synthesized, synthesized using in vitro transcription, or purified from cells. The latter approach may also reveal regions of the RNA that may be prevented from binding to oligonucleotides, e.g., by proteins, and may thus be less likely to be preferred regions to use in designing siRNAs. (See, e.g., Gunzl, A., et al, Methods, 26(2):162-9, Feb., 2002) Of course the general approach embodied in the foregoing method is not limited to RNase H but may employ any other nuclease that preferentially digests the RNA portion of a DNA/RNA hybrid. Enzymes that preferentially degrade or cleave double-stranded RNA while leaving single-stranded RNA intact (or vice versa), may be used in a similar fashion to identify preferred portions of the target (e.g., portions with a lesser propensity to assume secondary structures relative to other portions) for use in designing siRNAs.

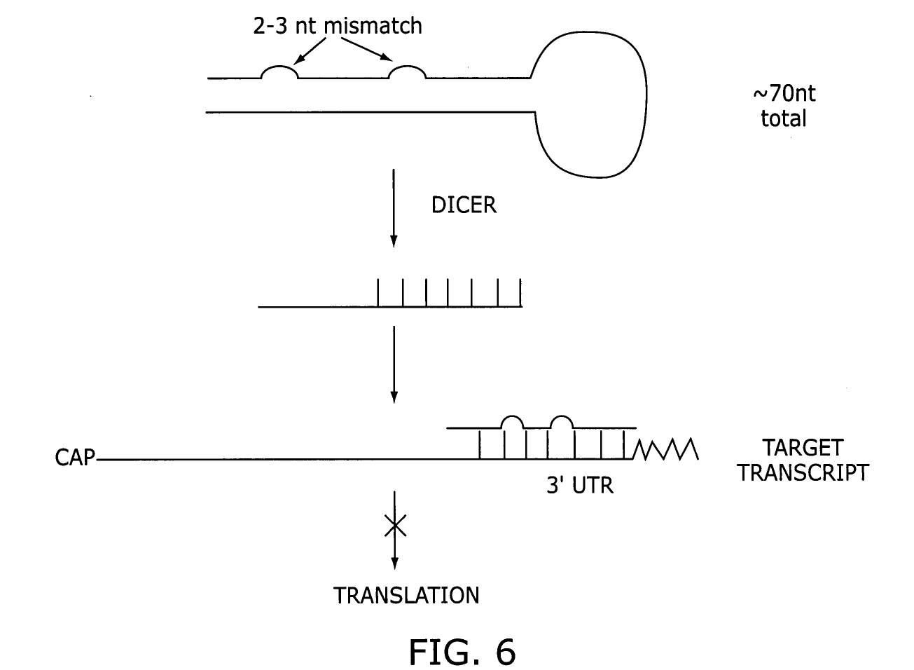

In some embodiments of the invention, the siRNA hybridizes to a target site that includes one or more 3′ UTR sequences. In fact, in certain embodiments of the invention, the siRNA hybridizes completely within the 3′ UTR. Such embodiments of the invention may tolerate a larger number of mismatches in the siRNA/template duplex, and particularly may tolerate mismatches within the central region of the duplex. In fact, some mismatches may be desirable as siRNA/template duplex formation in the 3′ UTR may inhibit expression of a protein encoded by the template transcript by a mechanism related to but distinct from classic RNA inhibition. In particular, there is some evidence to suggest that siRNAs that bind to the 3′ UTR of a template transcript may reduce translation of the transcript rather than decreasing its stability. Specifically, as shown in FIG. 6, the DICER enzyme that generates siRNAs in the Drosophila system discussed above and also in a variety of organisms, is known to also be able to process a small, temporal RNA (stRNA) substrate into an inhibitory agent that, when bound within the 3′ UTR of a target transcript, blocks translation of the transcript (see FIG. 6; Grishok, A., et al., Cell 106, 23-24, 2001; Hutvagner, G., et al., Science, 293, 834-838, 2001; Ketting, R., et al., Genes Dev., 15, 2654-2659.

Thus it is evident that a diverse set of RNA molecules containing duplex structures is able to mediate silencing through various mechanisms. For the purposes of the present invention, any such RNA, one portion of which binds to a target transcript and reduces its expression, whether by triggering degradation, by inhibiting translation, or by other means, is considered to be an siRNA, and any structure that generates such an siRNA (i.e., serves as a precursor to the RNA) is useful in the practice of the present invention.

In other embodiments of the invention, it may be desirable to design siRNAs targeted to 5′ untranslated regions of one or more transcripts. In particular, it may be desirable to target sequences such as the 5′ leader packaging sequence (see, for example, Chadwick et al., Gene Ther. 7:1362, 2000).

Those of ordinary skill in the art will readily appreciate that inventive siRNA agents may be prepared according to any available technique including, but not limited to chemical synthesis, enzymatic or chemical cleavage in vivo or in vitro, or template transcription in vivo or in vitro. As noted above, inventive siRNAs may be delivered as a single RNA strand including self-complementary portions, or as two (or possibly more) strands hybridized to one another. For instance, two separate 21 nt RNA strands may be generated, each of which contains a 19 nt region complementary to the other, and the individual strands may be hybridized together to generate a structure such as that depicted in FIG. 5A.

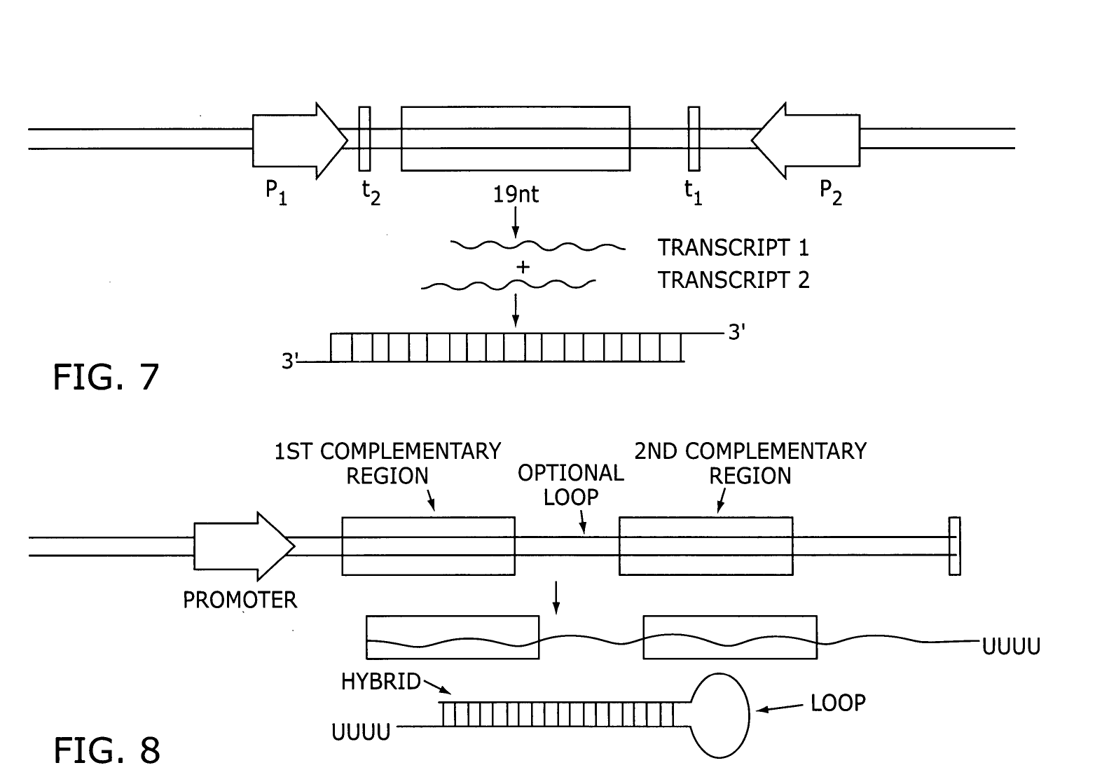

Alternatively, each strand may be generated by transcription from a promoter, either in vitro or in vivo. For instance, a construct (plasmid or other vector) may be provided containing two separate transcribable regions, each of which generates a 21 nt transcript containing a 19 nt region complementary with the other. Alternatively, a single construct may be utilized that contains opposing promoters (and, optionally, enhancers, terminators, and/or other regulatory sequences) positioned so that two different transcripts, each of which is at least partly complementary to the other, are generated is indicated in FIG. 7.

In another embodiment, an inventive siRNA agent is generated as a single transcript, for example by transcription of a single transcription unit encoding self complementary regions. FIG. 8 depicts one such embodiment of the present invention. As indicated, a template is employed that includes first and second complementary regions, and optionally includes a loop region. Such a template may be utilized for in vitro or in vivo transcription, with appropriate selection of promoter (and optionally other regulatory elements). The present invention encompasses gene constructs encoding one or more siRNA strands.

In vitro transcription may be performed using a variety of available systems including the T7, SP6, and T3 promoter/polymerase systems (e.g., those available commercially from Promega, Clontech, New England Biolabs, etc.). As will be appreciated by one of ordinary skill in the art, use of the T7 or T3 promoters typically requires an siRNA sequence having two G residues at the 5′ end while use of the SP6 promoter typically requires an siRNA sequence having a GA sequence at its 5′ end. Vectors including the T7, SP6, or T3 promoter are well known in the art and can readily be modified to direct transcription of siRNAs. When siRNAs are synthesized in vitro they may be allowed to hybridize before transfection or delivery to a subject. It is to be understood that inventive siRNA compositions need not consist entirely of double-stranded (hybridized) molecules. For example, siRNA compositions may include a small proportion of single-stranded RNA. This may occur, for example, as a result of the equilibrium between hybridized and unhybridized molecules, because of unequal ratios of sense and antisense RNA strands, because of transcriptional termination prior to synthesis of both portions of a self-complementary RNA, etc. Generally, preferred compositions comprise at least approximately 80% double-stranded RNA, at least approximately 90% double-stranded RNA, at least approximately 95% double-stranded RNA, or even at least approximately 99-100% double-stranded RNA.

Those of ordinary skill in the art will appreciate that, where inventive siRNA agents are to be generated in vivo, it is generally preferable that they be produced via transcription of one or more transcription units. The primary transcript may optionally be processed (e.g., by one or more cellular enzymes) in order to generate the final agent that accomplishes gene inhibition. It will further be appreciated that appropriate promoter and/or regulatory elements can readily be selected to allow expression of the relevant transcription units in mammalian cells.

In some embodiments of the invention in which inventive siRNAs are generated in vivo according to any of the approaches described above (e.g., using a single promoter, using two promoters, etc.), it may be desirable to utilize one or more regulatable promoter(s) or other regulatory sequences (e.g., inducible and/or repressible promoter); in other embodiments, constitutive expression may be desired. According to certain embodiments of the invention one or more of the regulatory sequences is tissue-specific and/or cell-type specific, so that the siRNA is produced in substantial amounts only in specific cells and/or tissues in which the promoter is active. For example, it may be desirable to utilize a promoter and/or enhancer that is active only in cells of the immune system, e.g., T cells, macrophages, etc. In some embodiments of the invention regulatory sequences may direct expression of a nucleotide sequence only in or at enhanced levels in cells that have been infected with HIV, relative to expression in cells not infected with HIV. For example, the regulatory sequence may comprise an HIV LTR element, a promoter containing a tat responsive element, etc. According to certain embodiments of the invention the construct comprises a nucleic acid sequence that encodes a selectable or detectable marker. Numerous such markers are known. For example, the construct may comprise an antibiotic resistance gene, a gene encoding a fluorescent molecule such as GFP, a gene encoding an enzyme such as β-galactosidase that catalyzes a chemical reaction to produce a readily detectable molecule, etc. Such markers are useful, for example, for selecting and/or isolating cells in which the construct is transcriptionally active (after, for example, contacting a population of cells with the construct). In the case of certain selectable markers, only cells in which the construct is transcriptionally active will survive under conditions of selection. In the case of detectable markers, cells in which the construct is transcriptionally active can be separated from cells that do not contain a transcriptionally active construct by any of a variety of means, e.g., FACS.

In certain preferred embodiments of the invention, the promoter utilized to direct in vivo expression of one or more siRNA transcription units is a promoter for RNA polymerase III (Pol III). Pol III directs synthesis of small transcripts that terminate within a stretch of 4-5 T residues. Certain Pol III promoters such as the U6 or H1 promoters do not require cis-acting regulatory elements (other than the first transcribed nucleotide) within the transcribed region and thus are preferred according to certain embodiments of the invention since they readily permit the selection of desired siRNA sequences. In the case of naturally occurring U6 promoters the first transcribed nucleotide is guanosine, while in the case of naturally occurring H1 promoters the first transcribed nucleotide is adenine. (See, e.g., Yu, J., et al., Proc. Natl. Acad. Sci., 99(9), 6047-6052 (2002); Sui, G., et al., Proc. Natl. Acad. Sci., 99(8), 5515-5520 (2002); Paddison, P., et al., Genes and Dev., 16, 948-958 (2002); Brummelkamp, T., et al., Science, 296, 550-553 (2002); Miyagashi, M. and Taira, K., Nat. Biotech., 20, 497-500 (2002); Paul, C., et al., Nat. Biotech., 20, 505-508 (2002); Tuschl, T., et al., Nat. Biotech., 20, 446-448 (2002). Thus in certain embodiments of the invention, e.g., where transcription is driven by a U6 promoter, the 5′-nucleotide of preferred siRNA sequences is G. In certain other embodiments of the invention, e.g., where transcription is driven by an HI promoter, the 5′ nucleotide may be A.

It will be appreciated that in vivo expression of constructs such as those depicted in FIGS. 7 and 8 can desirably be accomplished by introducing the constructs into a vector, such as, for example, a viral vector, and introducing the vector into mammalian cells. Any of a variety of vectors may be selected, though in certain embodiments it may be desirable to select a vector that can deliver the siRNA-encoding construct(s) to one or more cells that are susceptible to HIV infection. The present invention encompasses vectors containing siRNA transcription units, as well as cells containing such vectors or otherwise engineered to contain expressable transcription units encoding one or more siRNA strands. In certain preferred embodiments of the invention, inventive vectors are gene therapy vectors appropriate for the delivery of an siRNA-expressing construct to mammalian cells, preferably domesticated mammal cells, and most preferably human cells. Such vectors may be administered to a subject before or after exposure to HIV or a related virus (e.g., FIV, SIV) for prevention or treatment of HIV infection. Preferred gene therapy vectors include, for example, retroviral vectors and lentiviral vectors. In certain instances (e.g., gene therapy applications for HIV), lentiviruses will often be particularly preferred, due to their ability to infect resting T cells, dendritic cells, and macrophages. Lentiviral vectors can also transfer genes to hematopoietic stem cells with a superior gene transfer efficiency and without affecting the repopulating capacity of these cells. See, e.g., Mautino and Morgan, AIDS Patient Care STDS 2002 Jan;16(1):11-26. See also Lois, C., et al., Science, 295: 868-872, Feb. 1, 2002, describing the FUGW lentiviral vector; Somia, N., et al. J. Virol. 74(9): 4420-4424, 2000; Miyoshi, H., et al., Science 283: 682-686, 1999; and U.S. Pat. No. 6,013,516.

In certain embodiments of the invention two separate, complementary siRNA strands are transcribed using a single vector containing two promoters, each of which directs transcription of a single siRNA strand. In other embodiments of the invention a vector containing a promoter that drives transcription of a single siRNA strand comprising two complementary regions (e.g., a hairpin) is employed. In certain embodiments of the invention a vector containing multiple promoters, each of which drives transcription of a single siRNA strand comprising two complementary regions is used. Alternately, the vector may direct transcription of multiple different siRNAs, either from a single promoter or from multiple promoters. A variety of configurations are possible. For example, a single promoter may direct synthesis of a single RNA transcript containing multiple self-complementary regions, each of which may hybridize to generate a plurality of stem-loop structures. These structures may be cleaved in vivo, e.g., by DICER, to generate multiple different siRNAs. It will be appreciated that such transcripts preferably contain a termination signal at the 3′ end of the transcript but not between the individual siRNA units. It will be appreciated that single RNAs from which multiple siRNAs can be generated need not be produced in vivo but may instead be chemically synthesized or produced using in vitro transcription arid provided exogenously.

In another embodiment of the invention, the vector includes multiple promoters, each of which directs synthesis of a self-complementary RNA that hybridizes to form an siRNA. The multiple siRNAs may all target the same transcript, or they may target different transcripts. Any combination of viral and/or host cell transcripts may be targeted.

Those of ordinary skill in the art will further appreciate that in vivo expression of siRNAs according to the present invention may allow the production of cells that produce the siRNA over long periods of time (e.g., greater than a few days, preferably at least several months, more preferably at least a year or longer, possibly a lifetime). Such cells may be protected from HIV infection or replication indefinitely.

Inventive siRNAs may be introduced into cells by any available method. For instance, siRNAs or vectors encoding them can be introduced into host cells via conventional transformation or transfection techniques. As used herein, the terms “transformation” and “transfection” are intended to refer to a variety of art-recognized techniques for introducing foreign nucleic acid (e.g., DNA or RNA) into a host cell, including calcium phosphate or calcium chloride co-precipitation, DEAE-dextran-mediated transfection, lipofection, injection, or electroporation.

The present invention encompasses any cell manipulated to contain an inventive siRNA. Preferably, the cell is a mammalian cell, particularly human. Optionally, such cells also contain HIV RNA. In some embodiments of the invention, the cells are non-human cells within an organism. For example, the present invention encompasses transgenic animals engineered to contain or express inventive siRNAs. Such animals are useful for studying the function and/or activity of inventive siRNAs, and/or of the HIV infection/replication system. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the like. A transgene is exogenous DNA or a rearrangement, e.g., a deletion of endogenous chromosomal DNA, which preferably is integrated into or occurs in the genome of the cells of a transgenic animal. A transgene can direct the expression of an encoded siRNA product in one or more cell types or tissues of the transgenic animal. According to certain embodiments of the invention the transgenic animal is of a variety used as an animal model (e.g., murine or primate) for testing potential HIV therapeutics. Such models include primate models infected with SIV, murine models in which the immune system is reconstituted with human immune system cells, etc.

Identification of HIV Inhibitors

As noted above, the present invention provides a system for identifying siRNAs that are useful as inhibitors of HIV infection and/or replication. Specifically, the present invention demonstrates the successful preparation of siRNAs targeted to host genes or to viral genes to block or inhibit viral infection and/or replication. The techniques and reagents described herein can readily be applied to design potential new siRNAs, targeted to other genes or gene regions, and tested for their activity in inhibiting HIV infection and/or replication as discussed herein. As discussed herein, it is expected that HIV will continue to mutate and that it will always be desirable to develop and test new, differently targeted siRNAs, in some cases intended for administration to a single individual undergoing therapy.

Without wishing to be bound by any particular theory, we propose that it will often be desirable, when targeting viral genes, to target sequences present and available in internalized virus, e.g., uncoated virus (i.e., virus lacking the viral envelope) and/or de-encapsidated virus (e.g., prior to integration). It is appreciated, of course, that the ability to target internalized, uncoated, and/or de-encapsidated virus, rather than only later-generated transcripts containing the relevant sequence, may depend as much on the selected mode and timing of delivery as on the choice of sequence. Nonetheless, such targeting allows amplification of the inhibitory effect, through the early destruction of a first-level of RNA, which necessarily prevents production of downstream RNAs and progeny.

siRNAs that target pre-integrated virus (e.g., virus that has been internalized, uncoated, and/or de-encapsidated) can readily be identified as described herein. For instance, such agents are expected to have the same inhibitory effect on all viral RNAs, rather than discriminatory effects on individual transcripts.

In various embodiments of the invention potential HIV inhibitors can be tested by introducing candidate siRNA(s) into cells (e.g., by exogenous administration or by introducing a vector or construct that directs endogenous synthesis of siRNA into the cell) prior to, simultaneously with, or shortly after transfection with an HIV genome or portion thereof (e.g., within minutes, hours, or at most a few days) or prior to, simultaneously with, or shortly after infection with HIV. Alternately, potential HIV inhibitors can be tested by introducing candidate siRNA(s) into cells that are productively infected with HIV (i.e., cells that are producing progeny virus) or into cells that are latently infected with HIV (i.e., cells that contain a viral genome integrated into the host genome but are not producing progeny virus under the particular conditions employed). Latently infected cells may be stimulated to produce virus. The ability of the candidate siRNA(s) to reduce target transcript levels and/or to inhibit or suppress one or more aspects or features of the viral life cycle such as viral replication, pathogenicity, and/or infectivity is then assessed. For example, cell lysis, syncytia formation, production of viral particles, etc., can be assessed either directly or indirectly using methods well known in the art. Cells to which inventive siRNA compositions have been delivered (test cells) may be compared with similar or comparable cells that have not received the inventive composition (control cells). The susceptibility of the test cells to HIV infection can be compared with the susceptibility of control cells to infection. Production of viral protein(s) and/or progeny virus may be compared in the test cells relative to the control cells. Other indicia of viral infectivity, replication, pathogenicity, etc., can be similarly compared. Generally, test cells and control cells would be from the same species and of similar or identical cell type (e.g., T cell, macrophage, dendritic cell, etc.). For example, cells from the same cell line could be compared. When the test cell is a primary cell, typically the control cell would also be a primary cell. Typically the same HIV strain would be used to compare test cells and control cells.

In general, certain preferred HIV inhibitors reduce the target transcript level at least about 2 fold, preferably at least about 4 fold, more preferably at least about 8 fold, at least about 16 fold, at least about 64 fold or to an even greater degree relative to the level that would be present in the absence of the inhibitor (e.g., in a comparable control cell lacking the HIV inhibitor). In general, certain preferred HIV inhibitors inhibit entry of the infectious agent into the host cell by at least about 2 fold, preferably at least about 4 fold, more preferably at least about 8 fold, at least about 16 fold, at least about 64 fold or to an even greater degree relative to the extent of entry that would occur in the absence of the inhibitor (e.g., in a comparable control cell lacking the HIV inhibitor). In general, certain preferred HIV inhibitors inhibit HIV replication, so that the level of HIV replication is lower in a cell containing the inhibitor than in a control cell not containing the inhibitor by at least about 2 fold, preferably at least about 4 fold, more preferably at least about 8 fold, at least about 16 fold, at least about 64 fold or to an even greater degree. Similar considerations apply to testing potential inhibitors of other infectious agents.

Potential HIV inhibitors can also be tested using a variety of animal models (e.g., murine or primate) that have been developed. Compositions comprising candidate siRNA(s), constructs or vectors capable of directing synthesis of such siRNAs within a host cell, or cells engineered or manipulated to contain candidate siRNAs may be administered to an animal prior to, simultaneously with, or following infection with HIV (or an appropriate related virus in those models employing related viruses such as SIV). The ability of the composition to prevent HIV infection and/or to delay or prevent appearance of HIV-related symptoms and/or lessen their severity relative to HIV-infected animals that have not received the potential HIV inhibitor is assessed.

Analysis of HIV Infection/Replication

As noted above, one use for siRNAs of the present invention is in the analysis and characterization of the HIV infection/replication cycle. siRNAs may be designed that are targeted to any of a variety of host or viral genes involved in one or more stages of the viral infection and/or replication cycle. Such siRNAs may be introduced into cells prior to, during, or after HIV infection, and their effects on various stages of the infection/replication cycle may be assessed as desired. One feature of the present invention is its demonstration that host genes can be targeted to inhibit HIV infection and/or replication. The system can therefore be exploited to identify and/or characterize host genes that contribute to or participate in the viral life cycle. For instance, genes could be identified that protect from or participate in viral mutation. Those of ordinary skill in the art will immediately appreciate a wide range of additional or alternative applications.

Therapeutic Applications

Compositions containing inventive siRNAs of the present invention may be used to inhibit or reduce HIV infection or replication. In such applications, an effective amount of an inventive siRNA composition is delivered to a cell or organism prior to, simultaneously with, or after exposure to HIV. Preferably, the amount of siRNA is sufficient to reduce or delay one or more symptoms of HIV infection.

Inventive siRNA-containing compositions may contain a single siRNA species, targeted to a single site in a single target transcript, or alternatively may contain a plurality of different siRNA species, targeted to one or more sites in one or more target transcripts. In some embodiments of the invention, it will be desirable to utilize compositions containing collections of different siRNA species targeted to different genes. Some embodiments will include siRNAs targeted to both viral and host genes. Also, some embodiments will contain more than one siRNA species targeted to a single host or viral transcript. To give but one example, it may be desirable to include at least one siRNA targeted to coding regions of a target transcript and at least one siRNA targeted to the 3′ UTR. This strategy may provide extra assurance that products encoded by the relevant transcript will not be generated because at least one siRNA in the composition will target the transcript for degradation while at least one other inhibits the translation of any transcripts that avoid degradation. According to certain embodiments of the invention in which multiple transcripts are targeted, the transcripts include sequences from multiple different viral strains. These may include common variants and sequences associated with emergence of viral resistance. As is well known in the art, certain “escape” mutations are commonly found following anti-viral therapy and/or after culturing virus in vitro in the presence of anti-viral agents. Such mutations may be responsible for resistance, e.g., they may allow the encoded RNA or protein to function in the presence of the anti-viral agent. As described above, the invention encompasses such “therapeutic cocktails”, including approaches in which a single vector directs synthesis of siRNAs that inhibit multiple targets or of RNAs that may be processed to yield a plurality of siRNAs.

It is significant that the inventors have demonstrated effective siRNA-mediated inhibition of target transcript expression and of entry and replication of HIV using whole infectious virus as opposed, for example, to transfected genes, integrated transgenes, integrated viral genomes, infectious molecular clones, etc. In addition, it is of note that the inventors have demonstrated effective siRNA-mediated inhibition of HIV entry and infection using two different HIV strains. The R5 (BAL) and X4 (NL43) strains represent two major HIV strain variants, with R5 being macrophage-tropic and X4 being T cell-tropic. The demonstration that the same siRNA is effective against both of these major HIV variants is significant from a therapeutic standpoint.

It will be appreciated that HIV is well known for its mutability and therefore the emergence of resistance to therapeutic agents is a common problem. The emergence of resistance may be minimized by maintaining a low viral load (since low viral load implies fewer viruses and thus less total likelihood that a resistant variant will be produced). Attacking the virus at multiple points in the viral life cycle using a variety of siRNAs directed against host cell and/or viral transcripts presents an attractive approach to minimizing the emergence of resistant variants. Nevertheless, it is expected that, after an inventive composition has been administered to a cell infected with HIV, in some cases the virus may mutate so that it no longer is inhibited by the particular siRNA(s) provided. The present invention therefore contemplates evolving therapeutic regimes. In some cases, a preselected series of siRNAs, or combinations of siRNAs will be administered in a designated time course or in response to the evolution of resistance. In other cases, one or more new siRNAs can be selected in a particular case in response to a particular mutation. For instance, it would often be possible to design a new siRNA identical to the original except incorporating whatever mutation had been introduced into the virus; in other cases, it will be desirable to target a new sequence within the same gene; in yet other cases, it will be desirable to target a new gene entirely.

It will often be desirable to combine the administration of inventive siRNAs with one or more other anti-HIV agents in order to inhibit, reduce, or prevent one or more symptoms or characteristics of infection. In certain preferred embodiments of the invention, the inventive siRNAs are combined with approved agents such as those listed in Appendix B; however, the strategy may be utilized to combine the inventive siRNA compositions with one or more of any of a variety of agents including, for example, those listed in Appendix C.

In some embodiments of the invention, it may be desirable to target administration of inventive siRNA compositions to cells infected with HIV, or at least to cells susceptible of HIV infection (e.g., cells expressing CD4 including, but not limited to, immune system cells such as macrophages and T cells). Thus it is of note that the inventors have demonstrated effective siRNA-mediated suppression of expression of a target within T cells. In other embodiments, it will be desirable to have available the greatest breadth of delivery options.

As noted above, inventive therapeutic protocols involve administering an effective amount of an siRNA prior to, simultaneously with, or after exposure to HIV. For example, uninfected individuals may be “immunized” with an inventive composition prior to exposure to HIV; at risk individuals (e.g., prostitutes, IV drug users, or others who have recently experienced an exchange of bodily fluid with someone who is suspected, likely, or known to be infected with HIV) can be treated substantially contemporaneously with (e.g., within 48 hours, preferably within 24 hours, and more preferably within 12 hours of) a suspected or known exposure. Of course individuals known to be infected may receive inventive treatment at any time, including when viral load is undetectably low.

Gene therapy protocols may involve administering an effective amount of a gene therapy vector capable of directing expression of an inhibitory siRNA to a subject either before, substantially contemporaneously, with, or after HIV infection. Another approach that may be used alternatively or in combination with the foregoing is to isolate a population of cells, e.g., stem cells or immune system cells from a subject, optionally expand the cells in tissue culture, and administer a gene therapy vector capable of directing expression of an inhibitory siRNA to the cells in vitro either before or after expansion of the cells (typically before). A selection step may be employed to select cells that have taken up the gene therapy vector and/or in which it is transcriptionally active.

The cells may then be returned to the subject, where they may provide a population of HIV-resistant cells. Optionally, cells expressing the siRNA (which may thus become HIV-resistant) can be selected in vitro prior to introducing them into the subject. In some embodiments of the invention a population of cells, which may be cells from a cell line or from an individual who is not the subject, can be used. Methods of isolating stem cells, immune system cells, etc., from a subject and returning them to the subject are well known in the art. Such methods are used, e.g., for bone marrow transplant, peripheral blood stem cell transplant, etc., in patients undergoing chemotherapy.

In yet another approach, oral gene therapy may be used. For example, U.S. Pat. No. 6,248,720 describes methods and compositions whereby genes under the control of promoters are protectively contained in microparticles and delivered to cells in operative form, thereby achieving noninvasive gene delivery. Following oral administration of the microparticles, the genes are taken up into the epithelial cells, including absorptive intestinal epithelial cells, taken up into gut associated lymphoid tissue, and even transported to cells remote from the mucosal epithelium. As described therein, the microparticles can deliver the genes to sites remote from the mucosal epithelium, i.e. can cross the epithelial barrier and enter into general circulation, thereby transfecting cells at other locations.

Pharmaceutical Formulations

Inventive compositions may be formulated for delivery by any available route including, but not limited to parenteral (e.g., intravenous), intradermal, subcutaneous, oral (e.g., inhalation), transdermal (topical), transmucosal, rectal, and vaginal. Preferred routes of delivery include parenteral, transmucosal, rectal, and vaginal. Inventive pharmaceutical compositions typically include an siRNA or other agent(s) such as vectors that will result in production of an siRNA after delivery, in combination with a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use typically include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition should be sterile and should be fluid to the extent that easy syringability exists. Preferred pharmaceutical formulations are stable under the conditions of manufacture and storage and must be preserved against the contaminating action of microorganisms such as bacteria and fungi. In general, the relevant carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. Formulations for oral delivery may advantageously incorporate agents to improve stability within the gastrointestinal tract and/or to enhance absorption.

For administration by inhalation, the inventive siRNA agents are preferably delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit high therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

A therapeutically effective amount of a pharmaceutical composition typically ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The pharmaceutical composition can be administered at various intervals and over different periods of time as required, e.g., one time per week for between about 1 to 10 weeks, between 2 to 8 weeks, between about 3 to 7 weeks, about 4, 5, or 6 weeks, etc. For certain conditions such as HIV it may be necessary to administer the therapeutic composition on an indefinite basis to keep the disease under control. The skilled artisan will appreciate that certain factors can influence the dosage and timing required to effectively treat a subject, including but not limited to the severity of the disease or disorder, previous treatments, the general health and/or age of the subject, and other diseases present. Generally, treatment of a subject with an siRNA as described herein, can include a single treatment or, in many cases, can include a series of treatments.

Exemplary doses include milligram or microgram amounts of the inventive siRNA per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram.) It is furthermore understood that appropriate doses of an siRNA depend upon the potency of the siRNA, and may optionally be tailored to the particular recipient, for example, through administration of increasing doses until a preselected desired response is achieved. It is understood that the specific dose level for any particular animal subject may depend upon a variety of factors including the activity of the specific compound employed, the age, body weight, general health, gender, and diet of the subject, the time of administration, the route of administration, the rate of excretion, any drug combination, and the degree of expression or activity to be modulated.

The nucleic acid molecules of the invention can be inserted into vectors and used as gene therapy vectors as described herein. Gene therapy vectors can be delivered to a subject by, for example, intravenous injection, local administration, or by stereotactic injection (see e.g., Chen et al. (1994) Proc. Natl. Acad. Sci. USA 91:3054-3057). In certain embodiments of the invention gene therapy vectors may be delivered orally or inhalationally and may be encapsulated or otherwise manipulated to protect them from degradation, enhance uptake into tissues or cells, etc. The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral or lentiviral vectors, the pharmaceutical preparation can include one or more cells which produce the gene delivery system.

Inventive pharmaceutical compositions can be included in a container, pack, or dispenser together with instructions for administration.

### ADDITIONAL EMBODIMENTS

It will be appreciated that many of the teachings provided herein can readily be applied to infections with infectious agents other than HIV. The showing provided herein that host cell proteins and agent-specific proteins may effectively be targeted, resulting in a decrease in viral infectivity and/or replication or proliferation, clearly applies to any virus or other infectious agent that relies on the relevant host cell protein. The present invention therefore provides methods and compositions for inhibiting infection and/or replication by any infectious agent through administration of an siRNA agent that inhibits expression or activity of one or more host cell genes or agent-specific genes involved in the life cycle of the infectious agent.

Such conditions include those due to bacterial, viral, protozoal, and/or fungal agents. In each case, the skilled artisan will select one or more host transcripts (generally corresponding to host cell genes) necessary or important for effective infection, replication, survival, maturation, pathogenicity, etc., of the infectious agent and/or one or more agent-specific transcripts necessary or important for effective infection, survival, replication, maturation, etc., of the agent. By agent-specific transcript is meant a transcript having a sequence that differs from the sequence of transcripts normally found in an uninfected host cell. The agent-specific transcript may be present in the genome of the infectious agent or produced subsequently during the infectious process. One or more siRNAs will then be designed according to the criteria presented herein.

The ability of candidate siRNAs to suppress expression of target transcripts and/or the potential efficacy of the siRNA as a therapeutic agent may be tested using appropriate in vitro and/or in vivo (e.g., animal) models to select those siRNA capable of inhibiting expression of the target transcript(s) and/or reducing or preventing infectivity, pathogenicity, replication, etc., of the infectious agent. Appropriate models will vary depending on the infectious agent and can readily be selected by one of ordinary skill in the art. For example, for certain infectious agents and for certain purposes it will be necessary to provide host cells while in other cases the effect of siRNA on the agent may be assessed in the absence of host cells. As described above for HIV infection, siRNAs may be designed that are targeted to any of a variety of host or agent genes involved in one or more stages of the infection and/or replication cycle. Such siRNAs may be introduced into cells prior to, during, or after infection, and their effects on various stages of the infection/replication cycle may be assessed as desired.

Preferred host cell transcripts include, but are not limited to, transcripts that encode (1) receptors or other molecules that are necessary for or facilitate entry and/or intracellular transport of the infectious agent or a portion thereof such as the genome or proteins that produce or process such molecules; (2) cellular molecules that participate in the life cycle of the infectious agent, e.g., enzymes necessary for replication of the infectious agent's genome, enzymes necessary for integration of a retroviral genome into the host cell genome, cell signalling molecules that enhance pathogen entry and/or gene delivery, cellular molecules that are necessary for or facilitate processing of a viral component, viral assembly, and/or viral transport or exit from the cell. See, e.g., Greber, U., et al., “Signalling in viral entry”, Cell Mol Life Sci April 2002;59(4):608-26), Fuller A and Perez-Romero P, “Mechanisms of DNA virus infection: entry and early events”, Front Biosci Feb. 1, 2002;7:d390-406. Although host transcripts (generally corresponding to host cell genes) necessary or important for effective infection, replication, survival, maturation, pathogenicity, etc., of various infectious agents are known in the art and can be identified by reviewing the relevant scientific literature, additional such transcripts are likely to be identified in the future using any of a number of techniques. For example, candidates include host transcripts encoding molecules that physically associate with the infectious agent. The importance of a host transcript in the life cycle of an infectious agent may be determined by comparing the ability of the infectious agent to replicate or infect a host cell in the presence or absence of the host cell transcript. For example, cells lacking an appropriate receptor for an infectious agent would generally be resistant to infection with that agent.

Thus it is of note that the inventors have demonstrated effective siRNA-mediated suppression of expression of a host cell molecule (CD4), i.e., a molecule normally present in cells that are susceptible to infection by an infectious agent (HIV) and used as a receptor by the infectious agent and have acquired data suggesting that such suppression inhibited entry and replication of the infectious agent. In this regard it is of note that the inventors have demonstrated effective siRNA-mediated inhibition of target transcript expression and of entry and replication of an infectious agent using whole infectious virus as opposed, for example, to transfected genes, integrated transgenes, integrated viral genomes, infectious molecular clones, etc. The invention thus encompasses an siRNA targeted to a host cell transcript that is involved in replication, pathogenicity, or infection by an infectious agent and further encompasses methods of inhibiting replication, pathogenicity, or infection by an infectious agent by delivering siRNA to a cell susceptible to the agent. In certain preferred embodiments of the invention the siRNA inhibits expression of the host cell molecule in host cells that naturally express the gene as opposed, e.g., to cells that are engineered to express the molecule. In general, it is preferable to select cellular targets that are not required for essential activities of cells.

The invention further encompasses an siRNA targeted to an agent-specific transcript that is involved in replication, pathogenicity, or infection by an infectious agent. Preferred agent-specific transcripts that may be targeted in accordance with the invention include the agent's genome and/or any other transcript produced during the life cycle of the agent. Preferred targets include transcripts that are specific for the infectious agent and are not found in the host cell. For example, preferred targets may include agent-specific polymerases, sigma factors, transcription factors, etc. Such molecules are well known in the art, and the skilled practitioner will be able to select appropriate targets based on knowledge of the life cycle of the agent. In this regard useful information may be found in, e.g., Fields' Virology, 4^{th} ed., Knipe, D. et al. (eds.) Philadelphia, Lippincott Williams & Wilkins, 2001; Bacterial Pathogenesis, Williams, et al. (eds.) San Diego, Academic Press, 1998.

In some embodiments of the invention a preferred transcript is one that is particularly associated with the virulence of the infectious agent, e.g., an expression product of a virulence gene. Various methods of identifying virulence genes are known in the art, and a number of such genes have been identified. The availability of genomic sequences for large numbers of pathogenic and nonpathogenic viruses, bacteria, etc., facilitates the identification of virulence genes. Similarly, methods for determining and comparing gene and protein expression profiles for pathogenic and non-pathogenic strains and/or for a single strain at different stages in its life cycle agents enable identification of genes whose expression is associated with virulence. See, e.g., Winstanley, “Spot the difference: applications of subtractive hybridisation to the study of bacterial pathogens”, J Med Microbiol June 2002;51(6):459-67; Schoolnik, G, “Functional and comparative genomics of pathogenic bacteria”, Curr Opin Microbiol February 2002;5(1):20-6. For example, agent genes that encode proteins that are toxic to host cells would be considered virulence genes and may be preferred targets for siRNA. Transcripts associated with agent resistance to conventional therapies are also preferred targets in certain embodiments of the invention. In this regard it is noted that in some embodiments of the invention the target transcript need not be encoded by the agent genome but may instead be encoded by a plasmid or other extrachromosomal element within the agent.

In some embodiments of the invention the infectious agent is a drug-resistant bacterium. In some embodiments of the invention the infectious agent is a virus. In some embodiments of the invention the virus is a retrovirus or lentivirus. In certain embodiments of the invention the virus is a DNA virus. In some embodiments of the invention the virus is an RNA virus. In certain embodiments of the invention the virus is a virus other than a negative stranded RNA virus with a cytoplasmic life cycle, e.g., respiratory syncytial virus.

The siRNAs may have any of a variety of structures as described above (e.g., two complementary RNA strands, hairpin, structure, etc.). They may be chemically synthesized, produced by in vitro transcription, or produced within a host cell. The invention includes constructs and vectors capable of directing synthesis of the inventive siRNAs targeted to host cell transcript(s) or agent-specific transcript(s), cells containing such constructs or vectors, and methods of treatment in which the siRNAs, constructs, vectors, and/or cells are administered to a subject in need of treatment for or prevention of an infection.

### EXEMPLIFICATION

### EXAMPLE 1

### Transfection with CD4-siRNA Reduces CD4 Transcript Levels

The following Materials and Methods were employed in this and following Examples.

Cell Culture. Magi-CCR5 cells were grown in DMEM containing 200 ug/ml neomycin, 100 ug/ml hygromycin, and 10% heat-inactivated fetal calf serum (FCS). HeLa-CD4 cells were grown in DMEM containing 200 ug/ml neomycin and 10% heat-inactivated FCS.

Preparation of siRNAs. siRNAs with the following sense and antisense sequences were used (where the presence of a phosphate at the 5′ end of the RNA is indicated with a P):

| | | |
|---|---|---|
| CD4 (sense): | 5′-GAUCAAGAGACUCCUCAGUd | (SEQ ID NO:1) |
| | GdA-3′ | |
| CD4 | 5′-ACUGAGGAGUCUCUUGAUCd | (SEQ ID NO:2) |
| (antisense): | TdG-3′ | |
| p24 (sense): | 5′-P.GAUUGUACUGAGAGACAG | (SEQ ID NO:3) |
| | GCU-3′ | |
| p24 | 5′-P.CCUGUCUCUCAGUACAAU | (SEQ ID NO:4) |
| (antisense): | CUU-3′ | |
| GFP (sense): | 5′-P.GGCUACGUCCAGGAGCGC | (SEQ ID NO:5) |
| | ACC-3′ | |
| GFP | 5′-P.UGCGCUCCUGGACGUAGC | (SEQ ID NO:6) |
| (antisense): | CUU-3′ | |
| HPRT (sense) | 5′-P.GUGUCAUUAGUGAAACUG | (SEQ ID NO:7) |
| | GAA-3′ | |
| HPRT | 5′-P.CCAGUUUCACUAAUGACA | (SEQ ID NO:8) |
| (antisense) | CAA-3′ | |

All siRNAs were synthesized by Dharmacon Research (Lafayette, Colo.) using 2′ACE protection chemistry. The siRNA strands were deprotected according to the manufacturer's instructions, mixed in equimolar ratios and annealed by heating to 95° C. and slowly reducing the temperature by 1° C. every 30 s until 35° C. and 1° C. every min until 5° C.

siRNA transfection. Magi-CCR5 and HeLa cells were trypsinized and plated in 6 cm wells at 1×10⁵ cells per well for 12-16 h before transfection. Cationic lipid complexes, prepared by incubating 100 pmol of indicated siRNA with 3 ul oligofectamine (Gibco-Invitrogen, Rockville, Md.) in 100 ul DMEM (Gibco-Invitrogen) for 20 min, were added to the wells in a final volume of 1 ml. After overnight incubation, cells were washed and used for infection with HIV-1. For transfection of suspension cells, cationic lipid complexes were prepared by 20 min incubation with 100 pmol of indicated siRNA and 0.5 ul oligofectamine (Gibco-Invitrogen) in 50 ul AIM V T-cell medium (Gibco-Invitrogen). Log phase cultures of H9 cells were resuspended at 1×10⁵ cells per well in 50 ul AIM V media and combined with the cationic lipid complexes in 96 well flat bottom plates. Cells were transfected overnight, washed and resuspended in RPMI medium containing serum and were used for infection of HIV-1.

Flow cytometry. Phycoerythrin (PE)-conjugated αHIV-1 p24 monoclonal antibodies were used for staining (Shankar, P., et al., Blood 94, 3084-3093 (1999)). Data were acquired and analyzed on FACScalibur with CellQuest software (Becton Dickinson, Franklin Lakes, N.J.).

Northern Analysis. Northern blot analysis was performed with 5-10 ug total RNA (RNAEasy, Qiagen, Valencia, Calif.) and blotting was performed using the Northern Max protocol (Ambion, Austin, Tex.).

CD4 probe was PCR amplified from the T4pMV7 plasmid (Maddon, P. J., et al., Cell 47, 333-348 (1986)) using the following primers:

| | | |
|---|---|---|
| CD4-forward | 5′-TGAAGTGGAGGACCAGA | (SEQ ID NO:9) |
| | AGG-3′ | |
| CD4-reverse | 5′-CTTGCCCATCTGGAGGC | (SEQ ID NO:10) |
| | TTAG-3′ | |

p24 and nefprobes were PCR amplified from the HXB2 plasmid (Ratner, et al., AIDS Res. Hum. Retroviruses 3, 57-69 (1987) using the following primers:

| | | |
|---|---|---|
| p24-forward | 5′-CCAGGGGCAAATGGTACATCAGGCCATA-3′ | (SEQ ID NO:11) |
| p24-reverse | 5′-CCTCCTGTGAAGCTTGCTCGGCTCTTA-3′ | (SEQ ID NO:12) |
| nef-forward | 5′-ATGGGTGGCAAGTGGTCAAAAAGTAGTGTG-3′ | (SEQ ID NO:13) |
| nef-reverse | 5′-GTGGCTAAGATCTACAGCTGCCTTGTAAGT-3′ | (SEQ ID NO:14) |

β-actin probe (Ambion) was used as an internal standard. PCR products (25-30 ng) were labeled with α-[³²P]dATP (DECAprimell, Ambion), purified by NucAway spin columns (Ambion), heated to 95° C. and used as probes in Northern blots.

HIV infection. Magi-CCR5 cells were infected with R5 BAL and X4 NL43 strains of HIV-1 using 10 ng of p24 gag antigen per well. HeLa-CD4 cells were infected with 10-20 ng of p24 antigen per well of X4 HIVIIIB virus. At indicated times, cells were trypsinized and evaluated for HIV-1 p24 expression. H9 cells were infected with viral supernatants from pR7-GFP (Liu, R., et al., Cell 86, 367-377 (1996)) transfected 293 T cells at an MOI of 0.1.

β-gal staining. Magi-CCR5 cells were infected in the presence of DEAE-dextran (20 ug/ml) and then fixed and stained 2 d later (Chackerian, B., et al., J. Virol., 71, 3932-3939 (1997)). Cell counts represent number of blue cells per 10 high power fields. Cell-free p24 antigen was measured by ELISA in supernatants at indicated times (Beckman-Coulter, Brea, Calif.).

Results. To investigate the feasibility of using siRNA to suppress HIV replication, we targeted the CD4 molecule, the principal receptor for the virus (Klatzmann et al., Nature 312:767, 1984; Maddon et al., Cell 47:333, 1986). Specifically, we utilized the HeLa-derived cell line Magi-CCR5, which expresses human CD4, as well as CXCR4, the co-receptor for T-cell-tropic HIV, and CCR5, the co-receptor for macrophage-tropic virus (Chackerian et al., J. Virol. 71:3932, 1997). In addition, Magi-CCR5 cells have an integrated HIV-LTR-β-galactosidase gene that reflects Tat-mediated transactivation and can be used to score for viral entry and early gene expression.

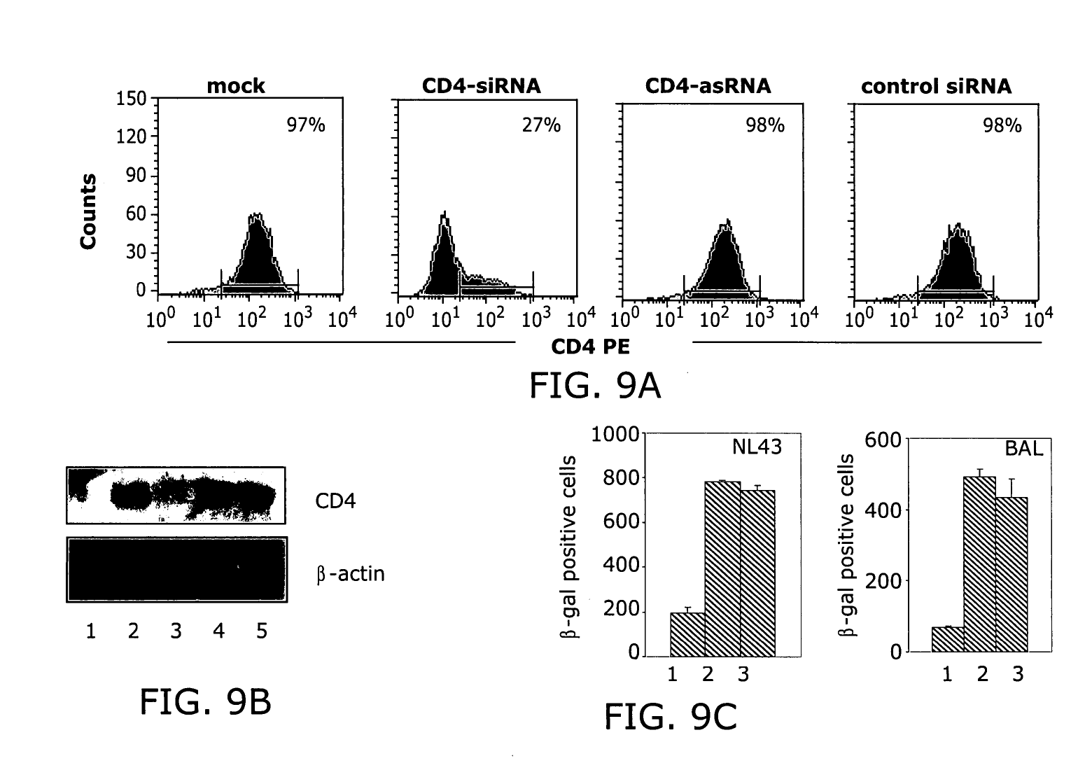

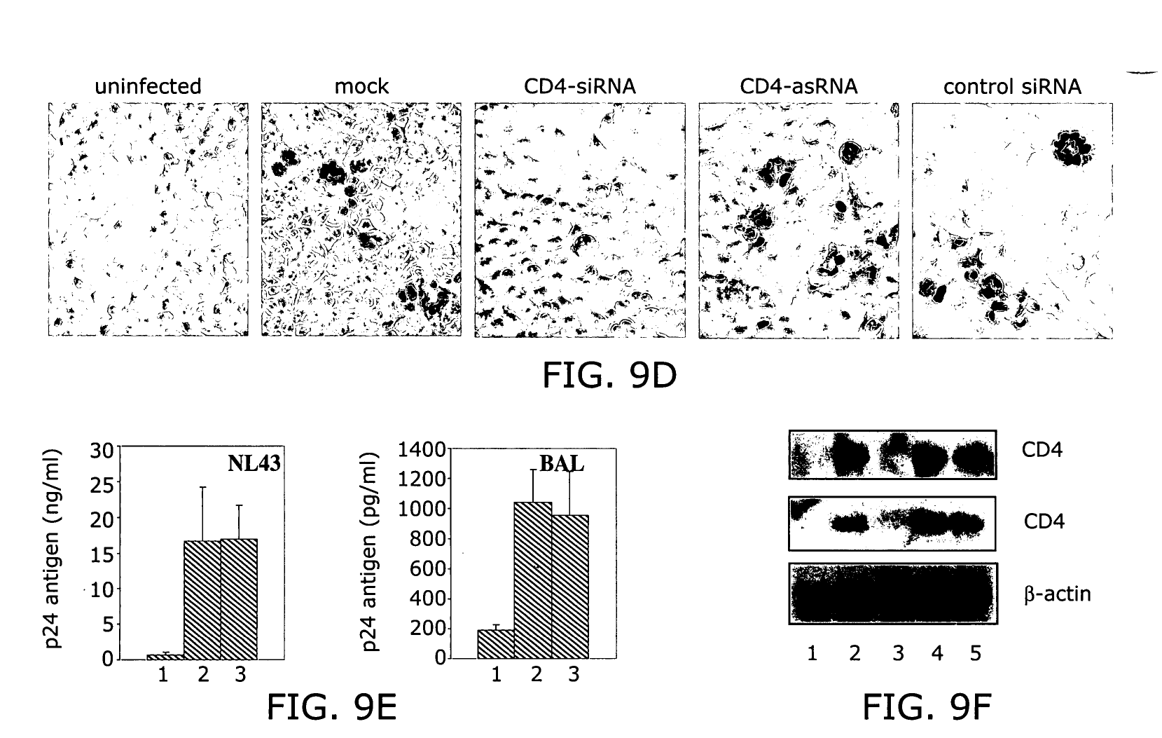

Magi-CCR5 cells were transfected either with siRNA directed against human CD4 or with control siRNA, and were analyzed for CD4 expression by flow cytometry. As shown in FIG. 9A, CD4-siRNA specifically reduced CD4 expression eight-fold in about 75% of the cells. Northern analysis, shown in FIG. 9B, revealed approximately an eight-fold reduction in CD4 mRNA, confirming that the CD4 silencing occurred at the level of mRNA stability. The exposure of the blot used for quantitation is shown in FIG. 9F.

### EXAMPLE 2

### CD4-siRNA Suppresses HIV Entry and Infection

To assess the effect of CD4 silencing on viral entry, Magi-CCR5 cells were first transfected with CD4-siRNA. Sixty hours later, the time of maximal gene silencing, the cells were infected with both R5 (BAL) macrophage tropic and X4 (NL43) T cell tropic strains of HIV. FIG. 9C shows the level of β-galactosidase activity observed 48 hours post-infection, which is an indicator of viral entry (cells expressing β-galactosidase appear dark in the figure); FIG. 9C shows the extent of syncytia formation, an indicator of viral infection. As can be seen, β-galactosidase levels were reduced 4-fold, and syncytia formation was almost abolished. Furthermore, early production of cell free virus, measured by p24 ELISA 48 hours post-infection, was reduced four-fold compared to cells treated with either antisense or control siRNA (see FIG. 9E). These findings, when taken together with those reported in Example 1, demonstrate that siRNA-directed silencing of CD4 specifically inhibited HIV entry into cells, and therefore blocked viral replication.

### EXAMPLE 3

### p24-siRNA Reduces Levels of p24 and of Viral Transcripts

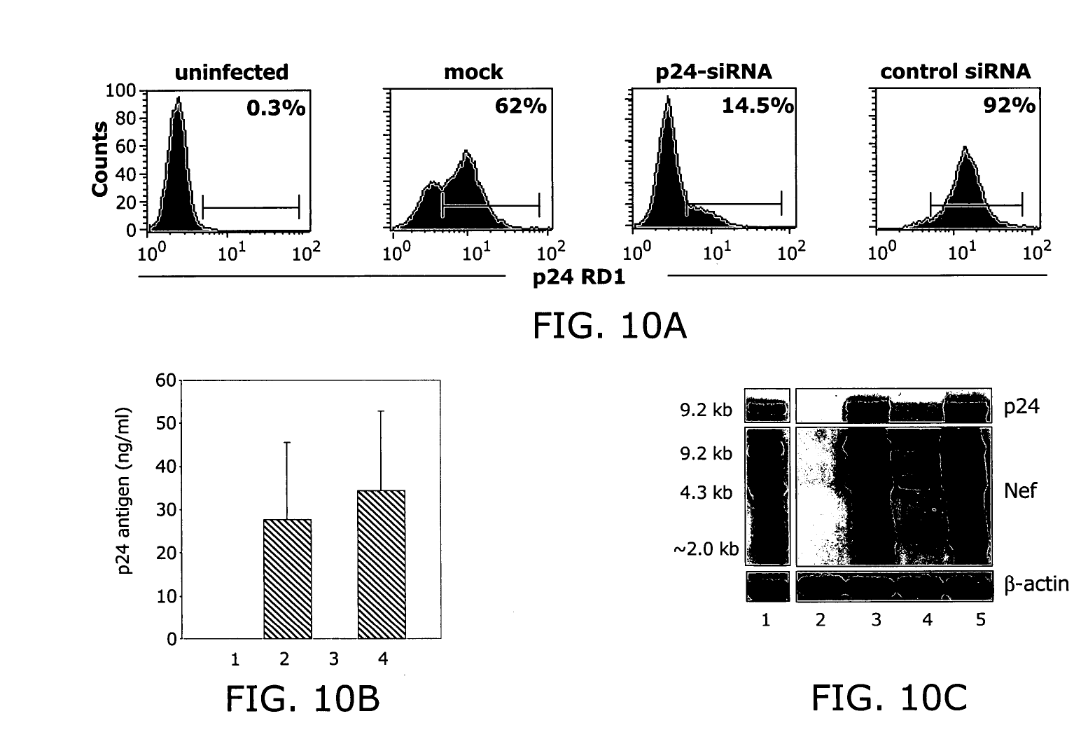

The HIV capsid is expressed from the intact viral RNA as a gag polyprotein that is proteolytically cleaved into p24, p17 and p15 polypeptides to form the major structural core of the virus. The p24 polypeptide also functions in uncoating and packaging virions. To score for siRNA-mediated HIV silencing of viral genes, we targeted the gag gene because cleavage in this region could inhibit both viral RNA accumulation and production of p24. HeLa cells expressing human CD4 (HeLa-CD4; Maddon et al., Cell 47:333, 1986) were transfected with p24-siRNA 24 hours prior to infection with HIVIIIB. Two days after infection, p24-siRNA transfected cells showed a greater than four-fold decrease in viral protein, compared with controls (FIG. 10A). Furthermore, silencing of full-length viral mRNA levels (as assessed by Northern blotting for p24 expression) was observed only in the p24-siRNA transfected HeLa-CD4 cells (FIG. 10B). Only 14.5% of p24-siRNA-transfected cells expressed p24 antigen above background levels 5 days after infection, while 92% of cells transfected with control siRNA had detectable p24 expression by flow cytometry (see FIG. 10C). When production of viral particules was measured by p24 ELISA 5 days after infection, p24 titers in culture supernatants were reduced 25-fold compared to mock transfected cells or cells transfected with control siRNA (see FIG. 10D). Northern blots of cellular RNA harvested 5 days after infection showed that after transfection with p24-siRNA, the amount of 9.2 Kd viral transcript containing gag p24 mRNA was reduced ten fold as compared with its level in control transfected cells (see FIG. 10E).

We also assessed the level of various HIV transcripts in the presence (or absence) of p24-siRNA. There are at least ten HIV transcripts (Pavlakis et al. in Ann. Rev. AIDS Res. (Kennedy et al., Eds) Marcel Dekker, New York: pp. 41-63, 1991), and multiple messenger RNAs—including several singly or multiply spliced messages, that are expressed from the integrated HIV provirus at various stages of the viral life cycle (Kim et al., J. Virol. 63:3708, 1989). The full-length HIV transcript is expressed only from the integrated provirus and serves as both the mRNA for the gag-pol genes and the genomic RNA of progeny virus. By contrast, some genes, including Tat, Rev, and Nef, may be expressed from the provirus prior to integration into the host genome (Wu et al., Science 293:1503, 2001).

Since Nef is the 3′-most gene and is contained in many virally-derived transcripts, a probe against Nef was used to test the effect of siRNA-directed knockdown on different viral transcripts. As shown in FIG. 10C, the 4.3 and 2.0 Kb Nef-containing transcripts were reduced approximately ten-fold, comparably to the knockdown of full-length transcript detected with p24 or Nef gene probes.

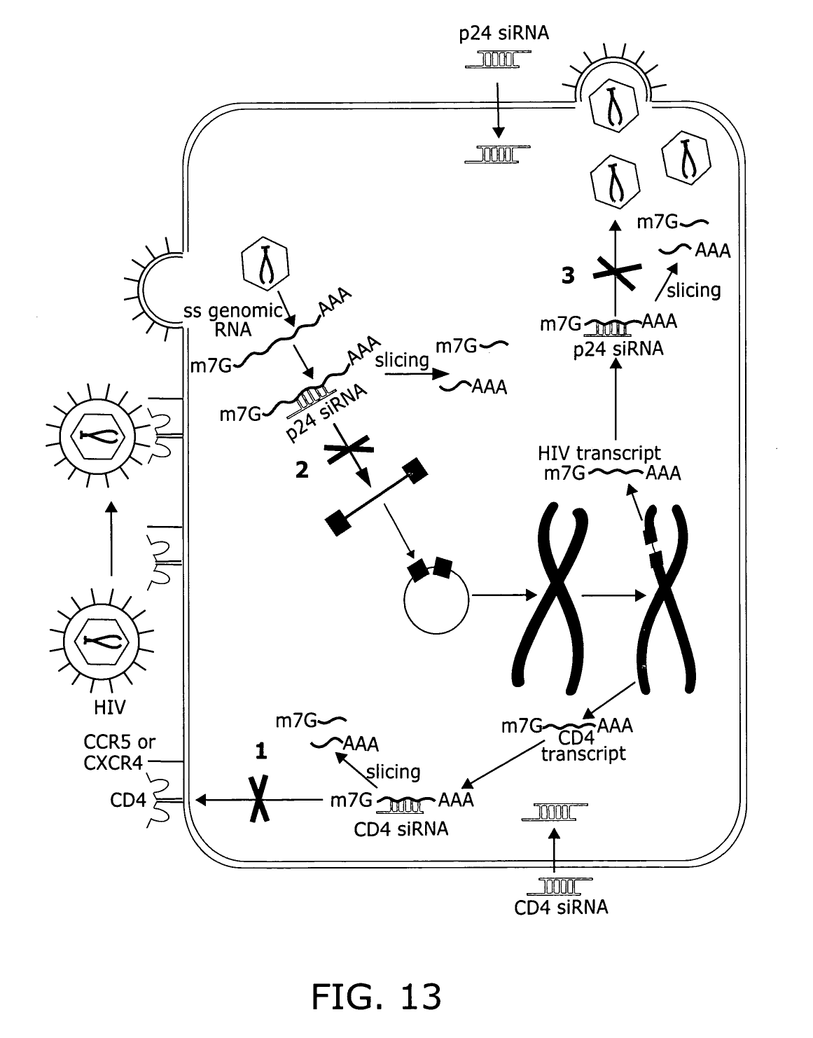

Mechanistically, these data suggest at least three possibilities: 1) the siRNA may target the viral genomic RNA directly when the virus first enters the cell, thereby affecting all subsequently-expressed HIV transcripts; 2) the siRNA may inhibit the pre-spliced mRNA in the nucleus; and/or 3) the siRNA may inhibit gag gene expression late in the viral life cycle either by targeting progeny viral genomes directly and/or by inhibiting viral capsid assembly, thereby blocking amplification and re-infection of the virus (see, for example, FIG. 13). Without wishing to be bound by any particular theory, we propose that the second possibility is least likely. In particular, we note intronic sequences have not been reported to be good targets for siRNA. Furthermore, Bitko and Barik have recently reported siRNA silencing of viral genes in mammalian cells infected with the respiratory syncytial virus (RSV) (BMC Microbiol. 34:1, 2001). Given that RSV not have a nuclear phase, it seems unlikely that the effects of siRNA could be attributed solely to inhibition of pre-spliced mRNAs in the nucleus. Consistent with this perspective, we note that the siRNA-containing RNA-induced silencing complex (RISC; Hammond et al., Nature 404:293, 2000) was isolated from ribosomal pellets of Drosophila cells (Hammond et al., Nature 404:293, 2000; Hammond et al., Science 293:1146, 2001). It is unlikely that this complex would have been found associated with ribosomes if it operated only in the nucleus.

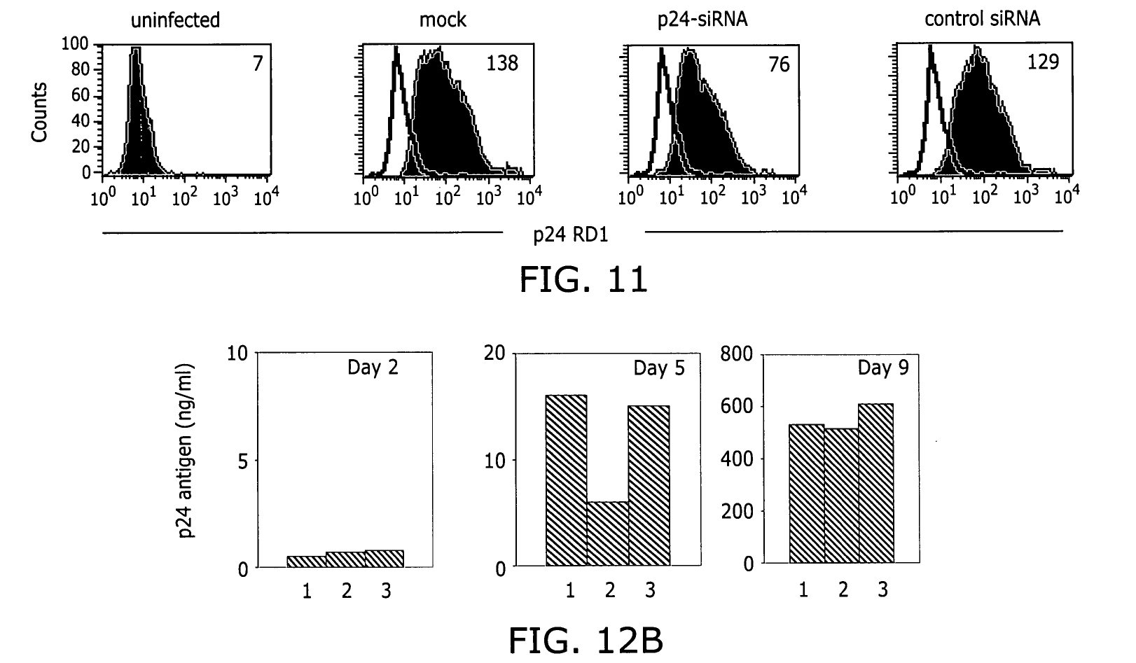

We further characterized the effects of p24-siRNA by asking whether this siRNA were able to suppress viral production post-integration. Specifically, we infected HeLa-CD4 cells with HIV four days prior to transfection with p24-siRNA. Two days after transfection, we assessed the mean fluorescent intensity of p24 expression on a per-cell basis. As shown in FIG. 11, we found that, in the setting of 80-90% HIV infection, mean fluorescent intensity of p24 expression was reduced 50% as compared with mock or control transfections. These results suggest that siRNA-directed silencing can reduce the steady-state levels of virus even in the setting of an established infection.

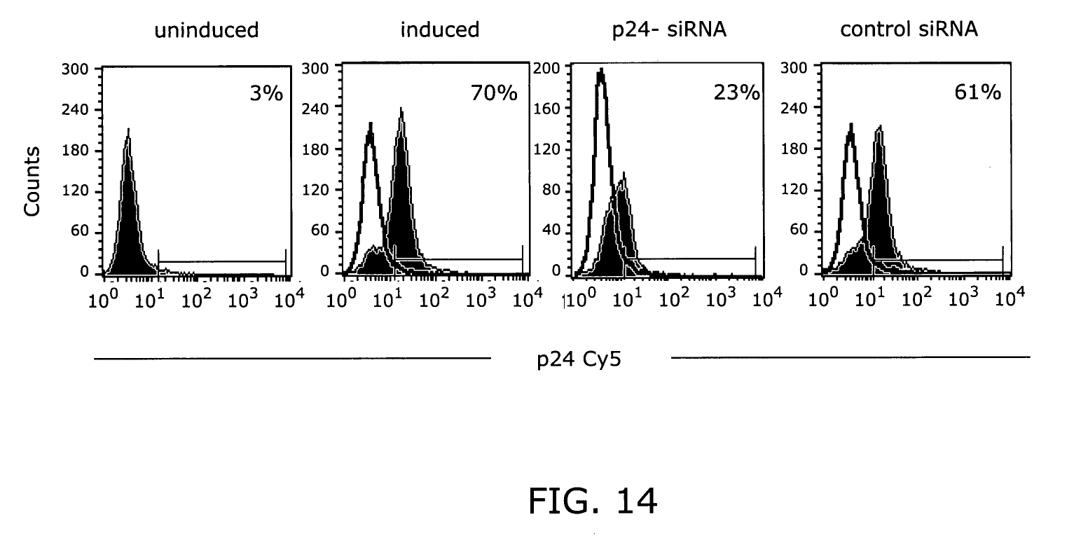

To further eliminate any potential effect of transfected siRNA on parental virus genomes before integration into the host genome, we assayed a latently infected T-cell clone (ACH2), which can be induced to produce high levels of infectious HIV-1 by phorbol myristate acetate (PMA) stimulation. ACH2 cells were grown in RPMI containing 10% heat-inactivated fetal calf serum. ACH2 cells were transfected with p24-siRNA and then induced by treating with PMA at 1 ug/ml. Two days after induction, 70% of control cells expressed p24 compared with 23% of the p24-siRNA-transfected cells (FIG. 14).

### EXAMPLE 4

### Time Course of siRNA Silencing of HIV Gene Expression

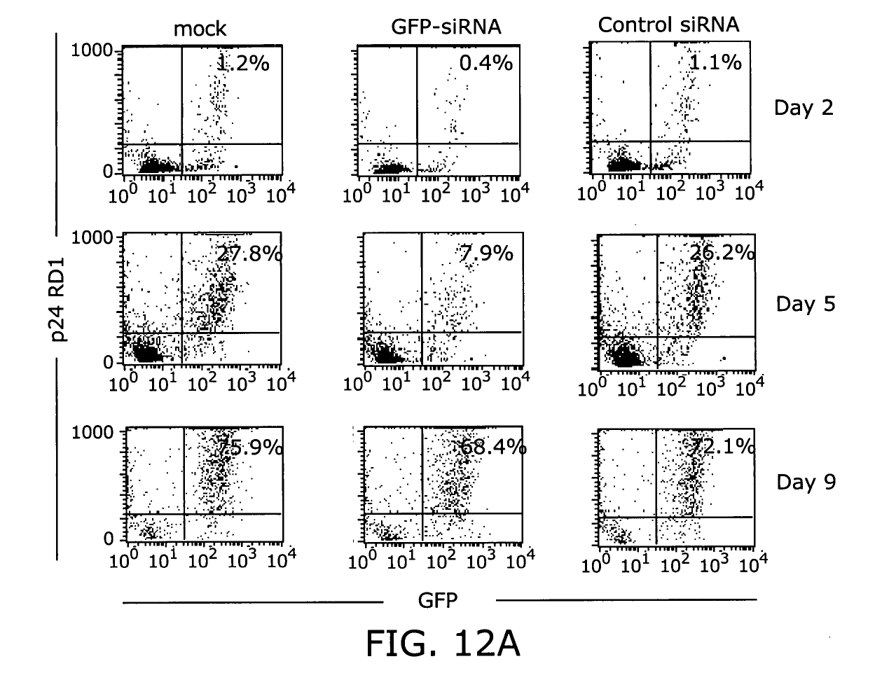

We also performed a time course of viral infectivity in a human T cell line. H9 cells transfected with GFP-siRNA were infected with an HIV strain in which the Nef gene had been replaced with GFP (Page, A., et al., AIDS Res. Hum. Retroviruses, 13, 1077-1081 (1997)). Two days after transfection, reduced levels of viral p24 and GFP proteins were detected (see FIG. 12). By day 5, HIV protein expression was still 3-4-fold lower than in control cells, but by day 9 post-transfection, the inhibition of viral production was minimal (see FIG. 12A). Similarly, p24 ELISA of culture supernatants revealed about three times less virus production by GFP-siRNA-transfected cells, as compared with control cells, five days after infection. However, after 9 days, the protective effect of siRNA was no longer detectable (see FIG. 12B). These results demonstrate viral inhibition beyond the time of maximal siRNA-directed gene silencing because inhibition of gene expression is maximal between 48-60 hours post-transfection and the wild-type level of gene expression is restored by 96 hours (not shown). Prolonged knockdown of viral gene expression is consistent with inhibition of viral amplification in multiple rounds of infection. Reduction of cell-free viral titers beyond the point of maximal viral gene silencing could reflect the siRNA-directed degradation of the viral genome at entry into the cell, or of the viral mRNAs transcribed from the integrated provirus.

We note that the reduction of cell-free virus titers observed in H9 cells (FIG. 12B) is less than the reduction observed in HeLa-CD4 cells (FIG. 10B). Transfection efficiency of siRNAs in HeLa cells is close to 100% as measured by reduction in CD4 levels, whereas the transfection efficiency is H9 cells is approximately 30% (data not shown). Therefore, amplification and re-infection is efficiently reduced in the HeLa-CD4 cells, but in H9 cells, approximately two thirds of the cells are poorly protected against the initial virus; such cells would be capable of progeny virus production and subsequent reinfection.

### EXAMPLE 5

### Inhibition of HIV Gene Expression in Primary T Cells.

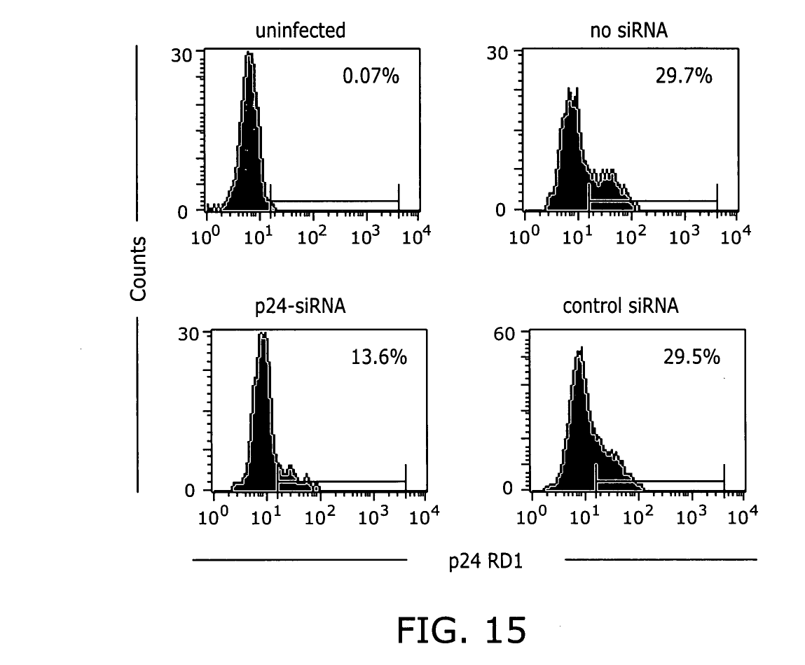

Inhibition of viral gene expression was also studied in primary T cells. CD4⁺ blasts were generated by isolating CD4⁺ T cells from peripheral blood lymphocytes of normal donors by immunomagnetic selection with Miltenyi beads (Miltenyi Biotech, Auburn, Calif.) and culturing them in RPMI 1640 containing 15% fetal calf serum in the presence of 4 μg/ml phytohemagglutinin (PHA). CD4⁺ cells activated with PHA for 4 days were mock, p24-siRNA, or GFP-siRNA (control siRNA) transfected. Twenty four hours later, the CD4⁺ blasts were infected with HIV_{IIIB}. Cells were analyzed 2 days later for p24 expression (p24-RD1) by flow cytometry. As shown in FIG. 15, inhibition of viral gene expression by siRNA-directed silencing in primary T cells was specific, although silencing of viral gene expression was only between 2- and 3-fold. Reduced siRNA-directed gene viral silencing in these cells may reflect either lower efficiency of silencing machinery or poor transfection efficiency in primary cells compared with cell lines. Nevertheless, these results demonstrate that the silencing machinery is active in primary cells and that inhibition of viral gene expression in primary cells can be achieved using siRNA.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the appended claims.

We claim:

1. A composition comprising:
an siRNA targeted to a target transcript, wherein the target transcript is a host cell transcript or agent-specific transcript, which transcript is involved in infection by or replication of an infectious agent.

2. The composition of claim 1, wherein the target transcript is a host cell transcript.

3. The composition of claim 1, wherein the target transcript is an agent-specific transcript.

4. The composition of claim 1, wherein:
the siRNA is present at a level sufficient to reduce the target transcript level at least about 2 fold.

5. The composition of claim 1, wherein:
the siRNA is present at a level sufficient to reduce the target transcript level at least about 4 fold.

6. The composition of claim 1, wherein:
the siRNA is present at a level sufficient to reduce the target transcript level at least about 8 fold.

7. The composition of claim 1, wherein:
the siRNA is present at a level sufficient to reduce the target transcript level at least about 16 fold.

8. The composition of claim 1, wherein:
the siRNA is present at a level sufficient to reduce the target transcript level at least about 64 fold.

9. The composition of claim 1, wherein:
the siRNA is present at a level sufficient to inhibit entry of the infectious agent into the host cell.

10. The composition of claim 9, wherein:
the siRNA is present at a level sufficient to inhibit entry of the infectious agent into the host cell by at least about 4 fold.

11. The composition of claim 9, wherein:
the siRNA is present at a level sufficient to inhibit entry of the infectious agent into the host cell by at least about 8 fold.

12. The composition of claim 9, wherein:
the siRNA is present at a level sufficient to inhibit entry of the infectious agent into the host cell by at least about 16 fold.

13. The composition of claim 9, wherein:
the siRNA is present at a level sufficient to inhibit entry of the infectious agent into the host cell by at least about 64 fold.

14. The composition of claim 1, wherein:
the target transcript is a host cell transcript that encodes a receptor for the infectious agent.

15. The composition of claim 1, wherein:
the target transcript is a host cell transcript that encodes a molecule that is not essential for cell survival or function.

16. The composition of claim 1, wherein:
the siRNA is present at a level sufficient to inhibit replication of the infectious agent.

17. The composition of claim 1, wherein:
the host cell is latently infected with the infectious agent, and the target transcript is an agent-specific transcript, and wherein the siRNA reduces expression of the target transcript.

18. The composition of claim 1, wherein:
presence of the siRNA in the host cell results in reduced levels of at least one agent-specific transcript other than the target transcript.

19. The composition of claim 1, wherein:
the siRNA comprises a base-paired region approximately 19 nucleotides long.

20. The composition of claim 1, wherein:
the siRNA comprises a base-paired region and at least one single-stranded overhang.

21. The composition of claim 1, wherein:
the siRNA comprises a hairpin structure.

22. The composition of claim 1, wherein:
the siRNA comprises a single RNA strand with a self-complementary region.

23. The composition of claim 1, wherein:
the siRNA comprises two complementary RNA strands.

24. The composition of claim 1, wherein:
the siRNA comprises a 3′ hydroxyl group.

25. The composition of claim 1, wherein:
the siRNA comprises a 5′ phosphate group.

26. The composition of claim 1, wherein:
the siRNA comprises a region that is precisely complementary with a region of the target transcript.

27. An analog of the siRNA of claim 1, wherein the analog differs from the siRNA in that it contains at least one modification.

28. The analog of claim 27, wherein:
the modification results in increased stability of the siRNA, enhances absorption of the siRNA, enhances cellular entry of the siRNA, or any combination of the foregoing.

29. The analog of claim 27, wherein:
the modification modifies a base, a sugar, or an internucleoside linkage.

30. An analog of the siRNA of claim 1, wherein:
the analog differs from the siRNA in that at least one ribonucleotide is replaced by a deoxyribonucleotide.

31. The composition of any of claims 1, 2, 3, or 27:
wherein the infectious agent is a virus.

32. The composition of claim 31 wherein:
the virus is a retrovirus or lentivirus.

33. The composition of claim 32, wherein:
the virus is HIV.

34. The composition of claim 1, wherein:
the host cell is an immune system cell.

35. The composition of claim 34, wherein:
the immune system cell is a T cell.

36. The composition of claim 1, wherein:
the host cell is a primary cell.

37. A composition comprising a plurality of single-stranded RNAs which, when hybridized, for m t the composition of claim 1.

38. The composition of claim 37, wherein:
the single-stranded RNAs range in length between approximately 21 and 23 nucleotides, inclusive.

39. The composition of claim 1, wherein:
the siRNA reduces the target transcript level without inducing an interferon response in the host cell.

40. The composition of claim 39, wherein:
the siRNA reduces the target transcript level without inducing an interferon response in the host cell under conditions in which an interferon response would be induced by introduction of a double-stranded RNA molecule into the host cell, wherein the double-stranded RNA molecule contains at least 30 base pairs.

41. An siRNA composition characterized in that when present within a cell susceptible to infection by HIV the composition reduces the susceptibility of the cell to infection by whole infectious HIV.

42. The siRNA composition of claim 41, the composition comprising double-stranded RNA.

43. The siRNA composition of claim 41, the composition comprising a vector that directs synthesis of siRNA.

44. The siRNA composition of claim 41, wherein:
the composition reduces the susceptibility of the cell to infection by at least two HIV strains.

45. The composition of claim 44, wherein:
the two strains include a T cell-tropic strain and a macrophage-tropic strain.

46. A composition comprising a nucleic acid construct, the construct characterized in that when present in a cell susceptible to infection by HIV, the construct directs transcription of one or more RNAs that reduce susceptibility of the cell to infection by whole infectious HIV.

47. An siRNA composition characterized in that when present within a cell infected by whole infectious HIV, the composition reduces viral protein production.

48. The siRNA composition of claim 47, the composition comprising double-stranded RNA.

49. The siRNA composition of claim 47, the composition comprising a vector that directs synthesis of siRNA.

50. The siRNA composition of claim 45, wherein:
the composition reduces the susceptibility of the cell to infection by at least two HIV strains.

51. The composition of claim 50, wherein:
the HIV strains include a T cell-tropic strain and a macrophage-tropic strain.

52. A pharmaceutical composition comprising:
the composition of claim 1; and
a pharmaceutically acceptable carrier.

53. A composition comprising a nucleic acid encoding an RNA operatively linked to expression signals active in a host cell so that, when the nucleic acid is introduced into the host cell, an siRNA is produced inside the host cell that is targeted to a host cell transcript or agent-specific transcript, which transcript is involved in infection by or replication of an infectious agent.

54. The composition of claim 53, wherein the infectious agent is a virus.

55. The composition of claim 54, wherein the virus is HIV.

56. The composition of claim 53, wherein:
the nucleic acid comprises a promoter for RNA polymerase III.

57. The composition of claim 56, wherein:
the promoter is a U6 or H1 promoter.

58. The composition of claim 53, wherein:
the nucleic acid comprises an inducible regulatory element.

59. The composition of claim 53, wherein:
the nucleic acid comprises a tissue or cell type specific regulatory element.

60. The composition of claim 53, wherein:
the nucleic acid comprises a regulatory element that direct expression of a nucleotide sequence only in or at enhanced levels in cells that have been infected with the infectious agent, relative to expression in cells not infected with the infectious agent.

61. A vector comprising the nucleic acid of claim 53.

62. The vector of claim 61, wherein:
the vector comprises a nucleic acid that encodes a selectable or detectable marker.

63. The vector of claim 61, wherein:
the vector is a vector suitable for gene therapy applications.

64. The vector of claim 63, wherein:
the vector is selected from the group consisting of retroviral vectors, lentiviral vectors, adenovirus vectors, and adeno-associated virus vectors.

65. A method of treating or preventing infection by an infectious agent, the method comprising steps of: administering to a subject prior to, simultaneously with, or after exposure of the subject to the infectious agent, a composition comprising the vector of claim 61.

66. The method of claim 65, wherein the infectious agent is a virus.

67. The method of claim 65, wherein the infectious agent is HIV.

68. A construct encoding one or both strands of an siRNA targeted to a transcript produced during infection by an infectious agent, which transcript is characterized in that its degradation delays, prevents, or inhibits one or more aspects of infection by or replication of the infectious agent.

69. A construct encoding one or both strands of an siRNA targeted to a transcript produced during HIV infection, which transcript is characterized in that its degradation delays, prevents, or inhibits one or more aspects of HIV infection or replication.

70. A vector comprising the construct of claim 68 or 69.

71. A cell engineered or manipulated to contain an siRNA targeted to a transcript produced during infection with an infectious agent, which transcript is characterized in that its degradation delays, prevents, or inhibits one or more aspects of infection by or replication of the infectious agent.

72. The cell of claim 71 wherein the infectious agent is a virus.

73. The cell of claim 72, wherein the virus is HIV.

74. A transgenic animal engineered to contain or express the siRNA composition of claim 1.

75. A method for identifying viral inhibitors, the method comprising steps of:
providing a cell including a candidate siRNA whose sequence includes a region of complementarity with at least one transcript produced during infection with a virus, which transcript is characterized in that its degradation delays, prevents, or inhibits one or more aspects of viral infection or replication;
detecting infection by or replication of the virus in the cell; and
identifying an siRNA that inhibits viral infectivity or replication, which siRNA is a viral inhibitor.

76. The method of claim 75, wherein:
the virus is HIV.

77. The method of claim 75, wherein:
the cell is characterized in that in the absence of the siRNA the cell produces at least one viral transcript.

78. The method of claim 75, wherein:
the cell is latently infected with the virus.

79. The method of claim 75, wherein:
the cell is productively infected with the virus.

80. The method of claim 75, further comprising the step of:
transfecting the cell with a viral genome or infecting the cell with the virus.

81. A method of treating or preventing infection by an infectious agent, the method comprising steps of:
administering to a subject prior to, simultaneously with, or after exposure of the subject to the infectious agent, a composition comprising an effective amount of an siRNA targeted to a transcript produced during infection by the infectious agent, which transcript is characterized in that reduction in levels of the transcript delays, prevents, or inhibits one or more aspects of infection by or replication of the infectious agent.

82. The method of claim 81, wherein:
the infectious agent is a virus.

83. The method of claim 82, wherein:
the virus is HIV.

84. A method of treating or preventing infection by an infectious agent, the method comprising administering to a subject prior to, simultaneously with, or after exposure of the subject to the infectious agent, a composition comprising an effective amount of an siRNA targeted to a transcript for a host cell gene, which transcript is characterized in that reduction in levels of the transcript delays, prevents, or inhibits one or more aspects of infection by or replication of the infectious agent.

85. The method of claim 84, wherein the infectious agent is a virus.

86. The method of claim 85, wherein the virus is a lentivirus or a retrovirus.

87. The method of claim 86, wherein the virus is HIV.

88. The method of claim 84, wherein the transcript encodes a receptor for the infectious agent.

89. A method of treating or preventing infection by an infectious agent, the method comprising administering to a subject prior to, simultaneously with, or after exposure of the subject to the infectious agent, a composition comprising the vector of claim 68 or a composition comprising the cell of claim 71.

90. A method of treating or preventing HIV infection, the method comprising administering to a subject prior to, simultaneously with, or after exposure of the subject to HIV, a composition comprising the vector of claim 69 or a composition comprising the cell of claim 73.

91. A method of treating or preventing HIV infection, the method comprising:
removing a population of cells from a subject at risk of or suffering from HIV infection;
engineering or manipulating the cells to contain an effective amount of an siRNA targeted to a transcript produced during HIV infection, which transcript is characterized in that its degradation delays, prevents, or inhibits one or more aspects of HIV infection or replication;
returning at least a portion of the cells to the subject.

92. A method of treating or preventing HIV infection, the method comprising:
removing a population of cells from a subject at risk of or suffering from HIV infection;
engineering or manipulating the cells to contain an effective amount of an siRNA targeted to a transcript for a host cell gene, which transcript is characterized in that reduction in levels of the transcript delays, prevents, or inhibits one or more aspects of HIV infection or replication;
returning at least a portion of the cells to the subject.

93. The method of claim 91 or 92, wherein:
the engineering or manipulating step comprises introducing a construct or vector that directs transcription of the siRNA into the cells.

94. The method of claim 93, wherein:
the siRNA comprises an RNA hairpin with a double-stranded portion.

95. The method of claim 93, wherein:
the siRNA comprises two complementary RNA strands.

96. The method of claim 91 or 92, wherein:
the cells comprise stem cells.

97. The method of claim 96, wherein:
the stem cells are peripheral blood stem cells.

98. The method of claim 91 or 92, further comprising:
selecting cells from the population that are not infected with HIV.

99. The method of claim 91 or 92, further comprising:
expanding at least a portion of the cells in culture.

100. The method of claim 91 or 92, wherein:
the cells returned to the subject in the returning step populate the immune system of the subject with HIV-resistant cells.
